(19) United States
(12) Patent Application Publication
(10) Pub. No.: US 2002/0156773 A1
(43) Pub. Date: Oct. 24, 2002

(54) Soluble HLA ligand database utilizing predictive algorithms and methods of making and using same

(76) Inventors: William Hildebrand, Edmond, OK (US); Kiley R. Prilliman, San Diego, CA (US); Heather D. Hickman, Oklahoma City, OK (US)

Correspondence Address: Dunlap, Codding & Rogers, P.C., Suite 420, 9400 North Broadway, Oklahoma City, OK, 73114, US

(21) Appl. No.: 10/082,034

(22) Filed: Feb. 21, 2002

Related U.S. Application Data
(63) Continuation-in-part of application No. 09/974,366, filed on Oct. 10, 2001.
(63) Continuation-in-part of application No. 10/022,066, filed on Dec. 18, 2001.
(60) Provisional application No. 60/270,357, filed on Feb. 21, 2001.

(51) Int. Cl. G06F 7/00

(52) U.S. Cl. 707/3

(57) ABSTRACT

The present invention generally relates to a MHC ligand database populated with MHC ligand sequences, motifs, extended motifs, submotifs, ligands unique to infected cells, tumor specific ligands, as well as a collection of current and future developed MHC ligand sequences developed by alternative methods. Other than the ligand sequences developed by alternative methods (which are in many cases non-standardized), the remaining ligand sequences are obtained in a standardized and minimum-variable dependent manner from soluble HLA molecules constructed according to the methodology described herein. The present invention further includes methodologies incorporating linear and predictive algorithm searching and comparison utilities.

### CROSS-REFERENCE TO RELATED APPLICATIONS

This application claims priority under 35 U.S.C. § 119(e) of provisional U.S. Serial No. 60/270,357, filed Feb. 21, 2001, entitled “SOLUBLE HLA LIGAND DATABASE AND EPITOPE PREDICATION SOFTWARE”, the contents of which are hereby expressly incorporated in their entirety by reference. This application is also a continuation-in-part of U.S. Ser. No. 09/974,366, filed Oct. 10, 2001, entitled “COMPARATIVE LIGAND MAPPING FROM MHC CLASS I POSITIVE CELLS;” and is also a continuation-in-part of U.S. Ser. No. 10/022,066, filed Dec. 18, 2001, entitled “METHOD AND APPARATUS FOR THE PRODUCTION OF SOLUBLE MHC ANTIGENS AND USES THEREOF,” the contents of which are also hereby expressly incorporated in their entirety by reference.

### BACKGROUND OF THE INVENTION

1. Field of the Invention

The present invention generally relates to a MHC ligand database populated with MHC ligand sequences, motifs, extended motifs, submotifs, ligands unique to infected cells, tumor specific ligands, as well as a collection of current and future developed MHC ligand sequences developed by alternative methods. Other than the ligand sequences developed by alternative methods (which are in many cases non-standardized), the remaining ligand sequences are obtained in a standardized and minimum-variable dependent manner from soluble HLA molecules constructed according to the methodology described herein. The present invention further includes methodologies incorporating linear and predictive algorithm searching and comparison utilities.

2. Brief Description of the State of the Art

There is a burgeoning volume of information and data arising from the rapid research and unprecedented progress in molecular biology. This has been particularly affected by the Human Genome Project which has apparently completely sequenced three billion nucleotides of the human genome. Other genome, protein, and other genetic sequencing projects are also contributing to this exponential growth in the number of genes and encoded proteins that have been sequenced. The number of journals, reports, research papers and tools required for the analysis of these sequences has also increased. For this reason the life sciences, and especially the field of immunology, requires tools in information technology and computation to prevent degradation of this data into an inchoate accretion of unconnected facts and figures.

Bioinformatics deals with organizing and presenting information in effective and meaningful ways. With the globalization of the Internet and the data deluge from the above-mentioned sequencing projects, bioinformatics is going through a period of explosive growth and development. The world wide web (“WWW”) facilitates the sharing of this information treasure trove and has changed the nature of learning by providing increased access to resources in a variety of media. According to the NCBI website (www.ncbi.nih.gov), bioinformatics is: “the field of science in which biology, computer science, and information technology merge into a single discipline. The ultimate goal of the field is to enable the discovery of new biological insights as well as to create a global perspective from which unifying principles in biology can be discerned. There are three important sub-disciplines within bioinformatics: (1) the development of new algorithms and statistics with which to assess relationships among members of large data sets; (2) the analysis and interpretation of various types of data including nucleotide and amino acid sequences, protein domains, and protein structures; and (3) the development and implementation of tools that enable efficient access and management of different types of information.”

The rationale for applying computational approaches to problems in biology include: (1) an explosive growth in the amount of biological information which necessitates the use of computers for information cataloging and retrieval; (2) a more global perspective in experimental design: as we move from the one scientist-one gene/protein/disease paradigm of the past to a consideration of whole organisms, we gain opportunities for new insights into health and disease; and (3) data mining—the process by which testable hypotheses are generated regarding the function or structure of a gene or protein of interest by identifying similar sequences in better characterized organisms. For example, new insight into the molecular basis of a disease may come from investigating the function of homologs of the disease gene in model organisms. Equally exciting is the potential for uncovering phylogenetic relationships and evolutionary patterns. With respect to the present invention, using knowledge gained regarding the sequences of individual peptides bound by a specific HLA allele along with derived motifs of all peptides bound by a specific HLA allele, scientists will be more capable to predict and/or identify novel epitopes for use in priming the immune response against pathogens such as HIV or hepatitis C as well as against tumors and/or autoimmune diseases.

The fields of immunology, vaccine development, and predictive modeling of immune system modulation are one such example of areas in which enormous amounts of sequencing and motif data are being generated daily. Unfortunately, the totality of this data is neither standardized nor normalized according to a set of controlled parameters or production procedures. Due to the previous unavailability of large quantities of purified and isolated HLA alleles, the sequencing of these alleles and the endogenously loaded peptide ligands presented thereby was a hit or miss proposition: numerous labs were attempting to sequence and identify peptide ligands presented and bound by an HLA molecule, yet none of these laboratories was capable of producing standardized information that was reproducible and presentable in an organized and meaningful manner.

Class I major histocompatibility complex (MHC) molecules, designated HLA class I in humans, bind and display peptide antigen ligands upon the cell surface. The peptide antigen ligands presented by the class I MHC molecule are derived from either normal endogenous proteins (“self”) or foreign proteins (“nonself”) introduced into the cell. Nonself proteins may be products of malignant transformation or intracellular pathogens such as viruses. In this manner, class I MHC molecules convey information regarding the internal fitness of a cell to immune effector cells including but not limited to, CD8⁺ cytotoxic T lymphocytes (CTLs), which are activated upon interaction with “nonself” peptides, thereby lysing or killing the cell presenting such “nonself” peptides.

Class II MHC molecules, designated HLA class II in humans, also bind and display peptide antigen ligands upon the cell surface. Unlike class I MHC molecules which are expressed on virtually all nucleated cells, class II MHC molecules are normally confined to specialized cells, such as B lymphocytes, macrophages, dendritic cells, and other antigen presenting cells which take up foreign antigens from the extracellular fluid via an endocytic pathway. The peptides they bind and present are derived from extracellular foreign antigens, such as products of bacteria that multiply outside of cells, wherein such products include protein toxins secreted by the bacteria that often times have deleterious and even lethal effects on the host (e.g. human). In this manner, class II molecules convey information regarding the fitness of the extracellular space in the vicinity of the cell displaying the class II molecule to immune effector cells, including but not limited to, CD4⁺ helper T cells, thereby helping to eliminate such pathogens. The elimination of such pathogens is accomplished by both helping B cells make antibodies against microbes, as well as toxins produced by such microbes, and by activating macrophages to destroy ingested microbes.

Class I and class II HLA molecules exhibit extensive polymorphism generated by systematic recombinatorial and point mutation events; as such, hundreds of different HLA types exist throughout the world's population, resulting in a large immunological diversity. Such extensive HLA diversity throughout the population results in tissue or organ transplant rejection between individuals as well as differing susceptibilities and/or resistances to infectious diseases. HLA molecules also contribute significantly to autoimmunity and cancer. Because HLA molecules mediate most, if not all, adaptive immune responses, large quantities of pure isolated HLA proteins are required in order to effectively study transplantation, autoimmunity disorders, and for vaccine development.

There are several applications in which purified, individual class I and class II MHC proteins are highly useful. Such applications include using MHC-peptide multimers as immunodiagnostic reagents for disease resistance/autoimmunity; assessing the binding of potentially therapeutic peptides; elution of peptides from MHC molecules to identify vaccine candidates; screening transplant patients for preformed MHC specific antibodies; linear and predictive algorithm databases containing sequences and motifs of peptide ligands bound by any one particular HLA allele; and removal of anti-HLA antibodies from a patient. Since every individual has differing MHC molecules, the testing of numerous individual MHC molecules is a prerequisite for understanding the differences in disease susceptibility between individuals. Therefore, purified MHC molecules representative of the hundreds of different HLA types existing throughout the world's population are highly desirable for unraveling disease susceptibilities and resistances, as well as for designing therapeutics such as vaccines.

Class I HLA molecules alert the immune response to disorders within host cells. Peptides, which are derived from viral-, bacterial- and tumor-specific proteins within the cell, are loaded into the class I molecule's antigen binding groove in the endoplasmic reticulum of the cell and subsequently carried to the cell surface. Once the class I HLA molecule and its loaded peptide ligand are on the cell surface, the class I molecule and its peptide ligand are accessible to cytotoxic T lymphocytes (CTL). CTL survey the peptides presented by the class I molecule and destroy those cells harboring ligands derived from infectious or neoplastic agents within that cell.

While specific CTL targets have been identified, little is known about the breadth and nature of ligands presented on the surface of a diseased cell. From a basic science perspective, many outstanding questions have permeated through the art regarding peptide exhibition. For instance, it has been demonstrated that a virus can preferentially block expression of HLA class I molecules from a given locus while leaving expression at other loci intact. Similarly, there are numerous reports of cancerous cells that fail to express class I HLA at particular loci. However, there are no data describing how (or if) the three classical HLA class I loci differ in the immunoregulatory ligands they bind. It is therefore unclear how class I molecules from the different loci vary in their interaction with viral-, bacterial and tumor-derived ligands and the number of peptides each will present.

Discerning virus-, bacteria- and tumor-specific ligands for CTL recognition is an important component of vaccine design. Ligands unique to tumorigenic or infected cells can be tested and incorporated into vaccines designed to evoke a protective CTL response. Several methodologies are currently employed to identify potentially protective peptide ligands. One approach uses T cell lines or clones to screen for biologically active ligands among chromatographic fractions of eluted peptides. (Cox et al., Science, vol 264, 1994, pages 716-719, which is expressly incorporated herein by reference in its entirety) This approach has been employed to identify peptides ligands specific to cancerous cells. A second technique utilizes predictive algorithms to identify peptides capable of binding to a particular class I molecule based upon previously determined motif and/or individual ligand sequences. (De Groot et al., Emerging Infectious Diseases, (7) 4, 2001, which is expressly incorporated herein by reference in its entirety) Peptides having high predicted probability of binding from a pathogen of interest can then be synthesized and tested for T cell reactivity in precursor, tetramer or ELISpot assays.

However, there has been no readily available source of individual HLA molecules for use in any of the aforementioned tests, experiments, and/or for the systematic and standardized isolation and sequencing of HLA peptide ligands and ligand motifs for use in populating an HLA ligand database. Such a database and coordinate linear and predictive algorithms which utilize significant quantities of data obtained through the standardized production, isolation and sequencing of endogenously loaded HLA peptide ligands as well as ligand motifs would be of immense value to those of ordinary skill in the art. The quantities of HLA protein previously available has been small and typically consisted of a mixture of different HLA molecules. Production of HLA molecules traditionally involved the growth and lysis of cells expressing multiple HLA molecules. Ninety percent of the population is heterozygous at each of the HLA loci; codominant expression results in multiple HLA proteins expressed at each HLA locus. To purify native class I or class II molecules from mammalian cells requires time-consuming and cumbersome purification methods, and since each cell typically expresses multiple surface-bound HLA class I or class II molecules, HLA purification results in a mixture of many different HLA class I or class II molecules. When performing experiments using such a mixture of HLA molecules or performing experiments using a cell having multiple surface-bound HLA molecules, interpretation of results cannot directly distinguish between the different HLA molecules, and one cannot be certain that any particular HLA molecule is responsible for a given result. Therefore, a need existed in the art for a method of producing substantial quantities of individual HLA class I or class II molecules so that they can be readily purified and isolated independent of other HLA class I or class II molecules.

Such individual HLA molecules, when provided in sufficient quantity and purity, provide a powerful tool for studying and measuring immune responses. Likewise, the accumulation of the data naturally evolving out of the isolation and sequencing of large pools of HLA ligands is of unique value to those of ordinary skill in the art. Indeed, such a database coupled with linear (such as BLAST searching, disclosed further hereinafter) and predictive algorithms (such as SYFPEITHI, Brown University's and Parker et al.'s algorithms, disclosed further hereinafter) results in a powerful, unique, and useful tool for those engaged in vaccine development and/or the basic research of the effect of HLA ligand binding on immune response.

Therefore, there exists a need in the art for improved methods for: the production of large quantities of HLA alleles and their endogenously loaded peptide ligands; the isolation and sequencing of endogenously loaded “self” peptides as well as the identification and sequencing of the endogenously loaded “nonself” peptides; isolation and sequencing of peptides presented by HLA in an infected or tumor cell but not in an uninfected tumor cell; methodology for the creation of motifs representative of the entire population of peptides endogenously loaded by a specific HLA allele in a normal cell, a pathogenic cell, and/or a tumor infected cell; and the creation of a centralized database populated with the ligand sequences and motifs so produced. The present invention solves this need by combining the production of soluble HLA molecules with an epitope isolation, discovery, and direct comparison methodology and with a soluble HLA ligand database into which the resulting data is accumulated and that also incorporates known and new searching and prediction capabilities—i.e. linear and predictive algorithm based searches.

### SUMMARY

The present invention generally relates to a MHC ligand database populated with MHC ligand sequences, motifs, extended motifs, submotifs, ligands unique to infected cells, tumor specific ligands, as well as a collection of current and future developed MHC ligand sequences developed by alternative methods. Other than the ligand sequences developed by alternative methods (which are in many cases non-standardized), the remaining ligand sequences are obtained in a standardized and minimum-variable dependent manner from soluble HLA molecules constructed according to the methodology described herein. The present invention further includes methodologies incorporating linear and predictive algorithm searching and comparison utilities.

In particular, the present invention provides for a method of accessing soluble HLA ligand data stored in a database. This method includes the following steps: (1) providing a database containing soluble HLA ligand data stored therein; (2) providing a means for accessing the database via a remote connection; and (3) providing a means for searching the soluble HLA ligand data stored in the database via the remote connection.

In another embodiment, the present invention provides for a computer system for a soluble HLA ligand database. This computer system includes: (1) a soluble HLA ligand database stored on memory media associated with the computer system, the soluble HLA ligand database having soluble HLA ligand data stored therein; and (2) a data retrieval process that includes instructions for: (a) receiving a request from a requestor for general soluble HLA ligand data and returning the retrieved data to the requestor, (b) receiving a match request from the requestor for soluble HLA ligand data and returning data that matches the match request to the requestor, and (c) receiving a predictive request from the requester for soluble HLA ligand data and returning data that matches the predictive request to the requestor.

In yet another embodiment of the present invention, the present invention provides for a soluble HLA ligand database assembled according to a methodology or process. This methodology or process includes the steps of: (1) providing a computer system capable of storing soluble HLA ligand data as a database on a memory media; (2) producing soluble HLA having ligands loaded thereon; (3) isolating the loaded ligands from the soluble HLA; (4) sequencing the loaded ligands to obtain soluble HLA ligand data; and (4) populating the database with the soluble HLA ligand data. The methodology or process may also further include the steps of linearly manipulating the soluble HLA ligand data in the database and/or the step of manipulating the soluble HLA ligand data in the database with a predictive algorithm.

### BRIEF DESCRIPTION OF THE SEVERAL VIEWS OF THE DRAWINGS

FIG. 1 is a graph of a reverse phase HPLC of the class I HLA B*150 and the eluted peptide ligands thereof.

FIG. 2 is a graphical representation of ion maps of peptides eluted from several B15 class I sHLA molecules.

FIG. 3 is a graphical representation of MS/MS fragmentation-sequencing of ion 517.2 from the B15 class I sHLA molecules referenced in FIG. 2.

FIG. 4 is an ion map of the peptides eluted from sHLA B*0702 in infected and uninfected cells.

FIG. 5 is a graphical representation of the deduced motifs and individual ligand sequences identified for three B15 class I sHLA molecules.

FIG. 6 is a graphical representation of a pooled peptide motif.

FIG. 7 is a graphical representation of submotifs for fractionated peptides.

FIG. 8 is a graphical representation of narrowing search parameters using fraction motifs for Ovarian Carcinoma Immunoreactive Antigen.

FIG. 9 is a MS graph showing the mass that corresponds with the ligand predicted by fraction 48 submotif seen in FIG. 8.

FIG. 10 is a graphical representation confirming that the peptide ligand predicted by a submotif is indeed present.

FIG. 11 is a tabular representation of motif data obtained by Edman sequencing.

FIG. 12 is a flow chart outlining the primary components of the sHLA ligand database of the present invention.

FIG. 13 is an Entity Relationship Diagram (ERD) for the sHLA ligand database of the present invention.

FIG. 14 is a UML diagram of the sHLA ligand database of the present invention.

### DETAILED DESCRIPTION OF THE INVENTION

Before explaining at least one embodiment of the invention in detail by way of exemplary drawings, experimentation, results, and laboratory procedures, it is to be understood that the invention is not limited in its application to the details of construction and the arrangement of the components set forth in the following description or illustrated in the drawings, experimentation and/or results. The invention is capable of other embodiments or of being practiced or carried out in various ways. Also, it is to be understood that the phraseology and terminology employed herein is for the purpose of description and should not be regarded as limiting. It should also be understood that the co-pending patent applications referenced hereinabove are expressly incorporated in their entirety as though fully recited within the body of the present specification.

The present invention generally defines a soluble HLA ligand database populated with ligand data derived according to the methodologies described herein as well as in the co-pending parent patent applications identified previously. The soluble HLA ligand database of the present invention contains data sets of amino acid sequences of: (1) individual endogenously loaded ligands for any specific HLA allele; (2) motifs (extended as well as submotifs) of endogenously loaded ligands; (3) endogenous ligands loaded uniquely in infected cells (viral or bacterial); and (4) ligands endogenously loaded uniquely in tumor or cancerous cells. The soluble HLA ligand database of the present invention relies upon a two-fold production, isolation, and sequencing methodology: (A) the production of large quantities of individual soluble HLA molecules having endogenously loaded peptides; and (B) the use of the individual soluble HLA molecules produced according to step A to discover/identify/sequence those individual peptides and/or peptide motifs bound by the soluble HLA molecule of interest to thereby create a cohesive, standardized and normalized data set of sHLA ligand information. This data set of sHLA ligand information is thereafter the core component in the sHLA ligand database of the present invention. The particularities of steps A and B are fully described in the co-pending parent applications which have been incorporated herein. For the sake of completeness, however, these steps are more generally described hereinafter.

### Production of sHLA Molecules with Endogenously Loaded Peptides

As mentioned previously, Class I major histocompatibility complex (MHC) molecules, designated HLA class I in humans, bind and display peptide antigens upon the cell surface. The peptides they present are derived from either normal endogenous proteins (“self”) or foreign proteins (“nonself”), such as products of malignant transformation or intracellular pathogens such as viruses. In this manner, class I molecules convey information regarding the internal fitness of a cell to immune effector cells including but not limited to CD8⁺ cytotoxic T lymphocytes (CTLs), which are activated upon interaction with “nonself” peptides and which lyse or kill the cell presenting such “nonself” peptides.

Class II MHC molecules, designated HLA class II in humans, also bind and display peptide antigens upon the cell surface. However, unlike class I MHC molecules which are expressed on virtually all nucleated cells, class II MHC molecules are normally confined to specialized cells, such as B lymphocytes, macrophages, dendritic cells, and other antigen presenting cells which take up foreign antigens from the extracellular fluid via an endocytic pathway. Therefore, the peptides they bind and present are derived from extracellular foreign antigens, such as products of bacteria that multiply outside of cells, wherein such products include protein toxins secreted by the bacteria that have deleterious and even lethal effects on the host. In this manner, class II molecules convey information regarding the fitness of the extracellular space in the vicinity of the cell displaying the class II molecule to immune effector cells including but not limited to CD4⁺ helper T cells, which help eliminate such pathogens both by helping B cells make antibodies against microbes as well as toxins produced by such microbes and by activating macrophages to destroy ingested microbes.

Characterizing naturally processed HLA class I and class II ligands is a key element behind the basic understanding of how polymorphism impacts ligand presentation. However, technical and scientific challenges including both extreme sample heterogeneity and limited sample sizes complicate such examinations. Thousands of distinct peptides are present within a ligand extract prepared from a single type of class I molecule, and the immunoprecipitation/extraction protocols typically employed to recover peptide ligands yield sparse quantities on the order of ˜20 Fg (Hunt et al. 1992; Henderson et al. 1993). These factors often require specialized biochemical expertise not necessarily available in either the common laboratory or core facility.

Other than the methodologies of the present invention, there is no readily available source of individual HLA molecules. Until now, the quantities of HLA protein available were small and typically consisted of a mixture of different HLA molecules. Production of HLA molecules traditionally involves growth and lysis of cells expressing multiple HLA molecules. Ninety percent of the population is heterozygous at each of the HLA loci; codominant expression results in multiple HLA proteins expressed at each HLA locus. To purify native class I or class II molecules from mammalian cells requires time-consuming and cumbersome purification methods, and since each cell typically expresses multiple surface-bound HLA class I or class II molecules, HLA purification results in a mixture of many different HLA class I or class II molecules. When performing experiments using such a mixture of HLA molecules or performing experiments using a cell having multiple surface-bound HLA molecules, interpretation of results cannot directly distinguish between the different HLA molecules, and one cannot be certain that any particular HLA molecule is responsible for a given result. Therefore, a need exists in the art for a method of producing substantial quantities of individual HLA class I or class II molecules so that they can be readily purified and isolated independent of other HLA class I or class II molecules. Such individual HLA molecules, when provided in sufficient quantity and purity, would provide a powerful tool for studying and measuring immune responses.

The present methodology provides a method of producing MHC molecules which are secreted from mammalian cells in a bioreactor unit. This methodology is detailed more explicitly in co-pending application U.S. Ser. No. 10/022,066 filed Dec. 18, 2001, which has been expressly incorporated herein. Substantial quantities of individual MHC molecules are obtained by modifying class I or class II molecules so they are secreted. Secretion of soluble MHC molecules overcomes the disadvantages and defects of the prior art in relation to the quantity and purity of MHC molecules produced. Problems of quantity are overcome because the cells producing the MHC do not need to be detergent lysed or killed in order to obtain the MHC molecule. In this way the cells producing secreted MHC remain alive and therefore continue to produce MHC. Problems of purity are overcome because the only MHC molecule secreted from the cell is the one that has specifically been constructed to be secreted. Thus, transfection of vectors encoding such secreted MHC molecules into cells which may express endogenous, surface bound MHC provides a method of obtaining a highly concentrated form of the transfected MHC molecule as it is secreted from the cells. Greater purity can be assured by transfecting the secreted MHC molecule into MHC deficient cell lines.

Production of the MHC molecules in a hollow fiber bioreactor unit allows cells to be cultured at a density substantially greater than conventional liquid phase tissue culture permits. Dense culturing of cells secreting MHC molecules further amplifies the ability to continuously harvest the transfected MHC molecules. Dense bioreactor cultures of MHC secreting cell lines allow for high concentrations of individual MHC proteins to be obtained. Highly concentrated individual MHC proteins provide an advantage in that most downstream protein purification strategies perform better as the concentration of the protein to be purified increases. Thus, the culturing of MHC secreting cells in bioreactors allows for a continuous production of individual MHC proteins in a concentrated form. cDNA or gDNA may be used as the starting material for the production of soluble HLA molecules. As will be appreciated, the use of gDNA has intrinsic advantages over the use of cDNA. As such, the use of gDNA as the starting material is preferred.

### Production of Soluble HLA (sHLA) from cDNA Starting Material

As shown in co-pending application U.S. Ser. No. 10/022,066, the initial HLA molecules selected for examination and production were from the HLA-B15 family. The HLA-B15 family represents a broad and diverse group of molecules comprised of nearly 50 evolutionarily related allotypes differing almost sequentially by 1-15 peptide binding groove residues, and they are observed throughout numerous ethnic populations (Hildebrand et al. 1994); serological and DNA-based typing thus far confirm distribution of B15 alleles among Caucasians, Amerindians (North and South), Mexicans, Blacks (African and American), Indians, Iranians, Pakistanis, Chinese, Japanese, Koreans, and Thais. The majority of HLA B-locus polymorphisms known to exist are represented among the members of this allelic family. HLA-B*1501 appears to be the “ancestral allele.”

The specific B15 allotypes initially selected for review and use with the production methodology were B*1501, B*1503, B*1508, B*1510, B*1512, and B*1518. B*1508 differs from B*1501 by a single mutagenic event in the α₁ helix, while B*1512 differs by a single mutagenic event in the α₂ helix; the remaining three alleles demonstrate a progressive series of polymorphisms throughout their binding grooves imposed by sequential mutagenic events during their divergent evolution from B*1501.

Using the primer sets of HLA5UT and sHLA3TM or 5PXI and 3PEI and template DNA from reliable full-length cDNA clones of HLA-B15 molecules B*1501, B*1503, B*1508, B*1510, B*1512, and B*1518, truncating PCR was performed for each on a Robocycler (Stratagene) for 30 cycles as described in Prilliman et al. 1997, which is expressly incorporated herein in its entirety. The resultant PCR products contained the leader peptide, α₁, α₂, and α₃ coding domains of the HLA heavy chain.

The PCR products were introduced into mammalian expression vectors. Initial constructs (truncated B*1501, B*1503, and B*1508) were prepared with the PSRα-neo vector (Lin et al. 1990), which has formerly been used to express non-truncated HLA molecules, while other constructs (truncated B*1501, B*1503, B*1508, B*1510, B*1512, and B*1518) were additionally prepared with either pcDNA3 or pcDNA3.1(-) (Invitrogen). Constructs using the PSRα-neo vector were made from PCR products of the HLA5UT and sHLA3TM primers; the PCR products were subcloned into M13 (mp18 or mp19) according to standard protocols so that confirmatory single-stranded DNA sequencing could be performed with Cy5-labelled versions of the primers M13 universal, 4N , and 3N (mp 18) or M13 universal, 3S , and JD3S (mp19), using the AutoLoad sequencing kit and ALFexpress automated sequencer (both Amersham Pharmacia Biotech). The insert was then prepared and purified, and this was followed by subcloning into PSRα-neo. Constructs using the pcDNA3 vector were made from PCR products of the 5PXI and 3PEI primers; these PCR products were subcloned into M13 and sequenced as above, following which the insert was subcloned into pcDNA3. Constructs using the pcDNA3.1(-) vector were made from products of the 5PXI and 3PEI primers; PCR products were directly subcloned into pcDNA3.1(-), following which double-stranded DNA sequence analysis was performed with Cy5-labelled versions of the primers 3S, 4N , T7 promoter, and pcDNA3.1/BGH.

DNA from each of the construct clones was prepared using Qiagen Midi kits for transfection of the class I-negative B-LCL 721.221. Cells growing in log phase in RPMI-1640+2 mM L-glutamine+ phenol red+ 20% FCS were pelleted and electroporation was performed prior to beginning selection with 1.5 mg/mL G418. Upon establishment of confluent growth after approximately 3 weeks, putative transfectant wells were screened for sHLA production using a sandwich ELISA. Transfectant wells positive for sHLA production were then subcloned by limiting dilution to establish cell lines optimally secreting greater than 1 Fg/mL of class I molecules in static culture over 48 h. Satisfactorily subcloned transfectants were then expanded, frozen in RPMI-1640+20% FCS+10% DMSO, and stored at −135 degrees C.

Since hollow-fiber bioreactors have been applied in place of in vivo hybridoma culture and monoclonal antibody (MAb) harvest from ascites in order to continuously produce large quantities of pure immunoglobulins, they were utilized to produce and harvest the sHLA of the present methodology. The Unisyn Technologies CP-3000 was selected for hollow-fiber bioreactor culture of successfully established transfectants. In this system, basal media is pumped into the fully-assembled system from a 200 L barrel; the media flows from the 4 L reservoir tank into the hollow-fiber networks of the four bioreactors, which provide 2.7 m² of surface area per cartridge, and then exits as waste. Extra capillary space (ECS) media and sHLA harvest are tandemly pumped into and out of the 270 mL cartridges, respectively, with each bioreactor receiving/yielding equal media/harvest volumes as regulated by inline solenoids.

The CP-3000 was set up according to the manufacturer's protocol. After the system was completely prepared, at least 1×10⁹ viable cells of a transfectant were grown in roller bottles of RPMI-1640+2 mM L-glutamine+phenol red+10% FCS. The cells were pelleted and inoculated into the ECS of the bioreactor cartridges. ECS feed and harvest bottles were then attached to their corresponding lines, and the basal and recirculation rates were initially set to 100 and 1000 mL/h, respectively; the ECS was usually not activated until 24-36 h following inoculation. The system was then monitored at least twice daily over 4-6 weeks, with adjustments made as necessary. This involved checking the glucose concentration and pH from manually-drawn reservoir samples, checking OD (optical density) readings, and regulating the basal and ECS rates accordingly. A fresh harvest sample was periodically extracted directly from the system to quantitate sHLA production by ELISA for production level monitoring.

Cells were cultured in the bioreactor system during each run until the desired amount of sHLA had been produced and collected in harvests. Each of the bioreactor runs was aborted once approximately 150 mg of sHLA was determined by ELISA to be contained in the respective harvests collected. The majority of runs were performed using 721.221 cells transfected with pcDNA3 or pcDNA3.1 (-) vector constructs, considering: (i) the significant differences in time frame between the runs performed using cell lines expressing soluble B*1501 from either the PSRα-neo or pcDNA3 vectors (3 months versus 1 month); and (ii) the fact that peptides extracted during each run produced identical motif results.

Upon completing a bioreactor run, sHLA complexes were purified from the harvests obtained. A 100 mL matrix of either the ρ₂m-specific MAb BBM.1 or W6/32 coupled to CNBr-activated Sepharose 4B (Amersham Pharmacia Biotech) according to the manufacturer's instructions was equilibrated with wash buffer (20 mM sodium phosphate, pH 7.2+0.02% sodium azide), and harvests were applied to the column using a GradiFrac LC system (Amersham Pharmacia Biotech); the load capacities for 100 mL matrices of the MAbs BBM.1 and W6/32 were approximated at 10 and 40 mg sHLA respectively, as monitored for saturation ELISA of screening pre- and post-column samples. The column was then washed, eluted with 0.2 N acetic acid, and neutralized with wash buffer. Both BBM.1 and W6/32 were used to affinity purify B*1501, the first molecule prepared. However, due to the differences in purification efficiency noted above between the two MAbs, W6/32 alone was employed to isolate the remaining molecules from bioreactor harvests. This MAb has been frequently used by others in purifying HLA.

The fractions collected during affinity column elution demonstrating UV absorbance at 280 nm were pooled, and glacial acetic acid was added to 10% volume to extract the peptides as described. Bound peptides were separated from heavy chains, β₂m, and BSA by passage through 3 kDa exclusion membrane filters (Amicon). The ligand-containing eluate was then lyophilized.

To remove residual salts and free amino acids remaining from the extraction process, isolated peptides were purified from free amino acids and salts prior to fractionation. This was done on a 2.1×100 mm C18 column (Vydac) with a steep RP-HPLC gradient using a DYNAMAX HPLC system (Rainin). The gradient was generated by increasing to 100% buffer B (0.06% TFA in 100% acetonitrile) in 1 min, holding for 10 min, and returning to buffer A (0.1% TFA in HPLC-grade water) in 1 min. The column was loaded with peptides reconstituted in the minimum volume of buffer A required for solubilization. During the run, the region corresponding to absorbance at 214 nm was manually collected. Typically 1/100th of the total purified ligand collection volume was removed and subjected to Edman sequencing for 14 cycles on a 492A pulsed liquid-phase protein sequencer (Perkin-Elmer Applied Biosystems Division) according to established protocols (Falk et al. 1991; Barber et al. 1995).

### Production of Soluble HLA (sHLA) from gDNA Starting Material

Another embodiment of the sHLA production methodology, as previously discussed herein-above, is the use of genomic DNA (gDNA) as the starting material for the production of the sHLA molecules.

This alternative method of the present invention begins by obtaining genomic DNA which encodes the desired MHC class I or class II molecule. Alleles at the locus which encode the desired MHC molecule are PCR amplified in a locus specific manner. These locus specific PCR products may include the entire coding region of the MHC molecule or a portion thereof. In some cases a nested or hemi-nested PCR is applied to produce a truncated form of the class I or class II gene so that it will be secreted rather than anchored to the cell surface. In other cases the PCR will directly truncate the MHC molecule.

Locus specific PCR products are cloned into a mammalian expression vector and screened with a variety of methods to identify a clone encoding the desired MHC molecule. The cloned MHC molecules are DNA sequenced to ensure fidelity of the PCR. Faithful truncated (i.e. sHLA) clones of the desired MHC molecule are then transfected into a mammalian cell line. When such cell line is transfected with a vector encoding a recombinant class I molecule, such cell line may either lack endogenous class I expression or express endogenous class I. It is important to note that cells expressing endogenous class I may spontaneously release MHC into solution upon natural cell death. In cases where this small amount of spontaneously released MHC is a concern, the transfected class I MHC molecule can be “tagged” such that it can be specifically purified away from spontaneously released endogenous class I molecules in cells that express class I molecules. For example, a DNA fragment encoding a His tail which will be attached to the protein may be added by the PCR reaction or may be encoded by the vector into which the gDNA fragment is cloned, and such His tail will further aid in purification of the class I molecules away from endogenous class I molecules. Tags beside a histidine tail have also been demonstrated to work and are logical to those skilled in the art of tagging proteins for downstream purification.

Cloned genomic DNA fragments contain both exons and introns as well as other non-translated regions at the 5′ and 3′ termini of the gene. Following transfection into a cell line which transcribes the genomic DNA (gDNA) into RNA, cloned genomic DNA results in a protein product thereby removing introns and splicing the RNA to form messenger RNA (mRNA), which is then translated into an MHC protein. Transfection of MHC molecules encoded by gDNA therefore facilitates reisolation of the gDNA, mRNA/cDNA, and protein.

Production of MHC molecules in non-mammalian cell lines such as insect and bacterial cells requires cDNA clones, as these lower cell types do not have the ability to splice introns out of RNA transcribed from a gDNA clone. In these instances the mammalian gDNA transfectants of the present invention provide a valuable source of RNA which can be reverse transcribed to form MHC cDNA. The cDNA can then be cloned, transferred into cells, and then translated into protein. In addition to producing secreted MHC, such gDNA transfectants therefore provide a ready source of mRNA, and therefore cDNA clones, which can then be transfected into non-mammalian cells for production of MHC. Thus, using a methodology which starts with MHC genomic DNA clones allows for the production of MHC in cells from various species.

Another key advantage of starting from gDNA is that viable cells containing the MHC molecule of interest are not needed. Since all individuals in the population have a different MHC repertoire, one would need to search more than 500,000 individuals to find someone with the same MHC complement as a desired individual—this is observed when trying to find a match for bone marrow transplantation. Thus, if it is desired to produce a particular MHC molecule for use in an experiment or diagnostic, a person or cell expressing the MHC allele of interest would first need to be identified. Alternatively, when using gDNA as the starting material, only a saliva sample, a hair root, an old freezer sample, or less than a milliliter (0.2 ml) of blood would be required to isolate the gDNA. Then, starting from gDNA, the MHC molecule of interest could be obtained via a gDNA clone as described, and following transfection of such clone into mammalian cells, the desired protein could be produced directly or in mammalian cells or from cDNA in several species of cells using the methods of the present invention described herein.

Current experiments to obtain an MHC allele for protein expression typically start from mRNA, which requires a fresh sample of mammalian cells that express the MHC molecule of interest. Working from gDNA does not require gene expression or a fresh biological sample. It is also important to note that RNA is inherently unstable and is not easily obtained as is gDNA. Therefore, if production of a particular MHC molecule starting from a cDNA clone is desired, a person or cell line that is expressing the allele of interest must traditionally first be identified in order to obtain RNA. Then a fresh sample of blood or cells must be obtained; experiments using the methodology of the present invention show that≧5 milliliters of blood that is less than 3 days old is required to obtain sufficient RNA for MHC cDNA synthesis. Thus, by starting with gDNA, the breadth of MHC molecules that can be readily produced is expanded. This is a key factor in a system as polymorphic as the MHC system; hundreds of MHC molecules exist, and not all MHC molecules are readily available from MRNA. This is especially true of MHC molecules unique to isolated populations or of MHC molecules unique to ethnic minorities. Starting class I or class II protein expression from the point of genomic DNA simplifies the isolation of the gene of interest and ensures a more equitable means of producing MHC molecules for study; otherwise, one would be left to determine whose MHC molecules are chosen and not chosen for study, as well as to determine which ethnic population from which fresh samples cannot be obtained should not have their MHC molecules included in a diagnostic assay.

While cDNA may be substituted for genomic DNA as the starting material, production of cDNA for each of the desired HLA class I types will require hundreds of different, HLA typed, viable cell lines, each expressing a different HLA class I type. Alternatively, fresh samples are required from individuals with the various desired MHC types. The use of genomic DNA as the starting material allows for the production of clones for many HLA molecules from a single genomic DNA sequence, as the amplification process can be manipulated to mimic recombinatorial and gene conversion events. Several mutagenesis strategies exist whereby a given class I gDNA clone could be modified at either the level of gDNA or cDNA resulting from this gDNA clone. The process of the present invention does not require viable cells, and therefore the degradation which plagues RNA is not a problem. Thus, from a given gDNA clone, any number of gDNA and cDNA MHC molecules can be produced.

Three useful products can be obtained from the mammalian cell line expressing HLA class I molecules from such a genomic DNA construct. The first product is the soluble class I MHC protein, which may be purified and utilized in various experimental strategies, including but not limited to epitope testing. Epitope testing is a method for determining how well discovered or putative peptide epitopes bind individual, specific class I or class II MHC proteins. Epitope testing with secreted individual MHC molecules has several advantages over the prior art, which utilized MHC from cells expressing multiple membrane-bound MHCs. While the prior art method could distinguish if a cell or cell lysate would recognize an epitope, such method was unable to directly distinguish in which specific MHC molecule the peptide epitope was bound. Lengthy purification processes might be used to try and obtain a single MHC molecule, but doing so limits the quantity and usefulness of the protein obtained. The novelty of the current approach is that individual MHC specificities can be utilized in sufficient quantity through the use of recombinant, soluble MHC proteins. Because MHC molecules participate in numerous immune responses, studies of vaccines, transplantation, immune tolerance, and autoimmunity can all benefit from individual MHC molecules provided in sufficient quantity.

A second important product obtained from mammalian cells secreting individual MHC molecules is the peptide cargo carried by MHC molecules. Class I and class II MHC molecules are really a trimolecular complex consisting of an alpha chain, a beta chain, and the alpha/beta chain's peptide cargo to be reviewed by immune effector cells. Since it is the peptide cargo, and not the MHC alpha and beta chains, which marks a cell as infected, tumorigenic, or diseased, there is a great need to characterize the peptides bound by particular MHC molecules. For example, characterization of such peptides will greatly aid in determining how the peptides presented by a person with MHC-associated diabetes differ from the peptides presented by the MHC molecules associated with resistance to diabetes. As stated above, having a sufficient supply of an individual MHC molecule, and therefore that MHC molecule's bound peptides, provides a means for studying such diseases. Because the method of the present invention provides quantities of MHC protein previously unobtainable, unparalleled studies of MHC molecules and their important peptide cargo can now be facilitated.

The methodology for producing sHLA from gDNA, while similar to the methodology for producing sHLA from cDNA, is different and as such requires different and/or unique steps and/or processes for its completion. One exemplary detailed production methodology for using gDNA as the starting material for the production of MHC class I or II molecules is described in co-pending U.S. application Ser. No. 10/022,066, which has been expressly incorporated herein by reference.

Due to the polymorphic nature of the HLA system, production of many alleles as soluble molecules is very difficult as a viable cell line is required in order to make cDNA, and quite often this is not available. Thus a method that allows us to produce many different alleles from a readily available starting point is invaluable. Production of the sHLA from genomic DNA provides such a starting point. We have shown here that two simple PCR reactions allows us to clone many, if not all, HLA Class I alleles from genomic DNA.

The Elisa data allows us to test how functional these molecules are. By using W6/32 and anti β₂M to establish production levels, we also provide information as to how much of the protein is in a trimeric form. The comparative Elisa data helps back this up as the ratio of W6/32:HC10 needs to be greater than 1.0 in order for there to be more conformational molecule than denatured, this is shown to be the case. In summary we have developed a technique that will allow for the production of virtually any HLA Class I molecule in a soluble form on demand.

An exemplary useful product which can be obtained from the mammalian cell line expressing such a genomic DNA construct is a cDNA clone encoding the desired class I or class II molecule. The cDNA clone encoding the desired class I or class II molecule is formed from the mRNA molecule encoding the desired class I molecule isolated from such mammalian cell line. The cDNA clone may be utilized for functional testing. Thus, gDNA clones can be used as a mechanism to obtain cDNA clones of the desired class I or class II HLA molecule.

The CDNA clones may be transfected into a cell which is unable to splice introns and process the mRNA molecule and therefore would not express the MHC molecule encoded by the genomic DNA, such as insect cells or bacterial cells. In addition, these cell lines will also be deficient in peptide processing and loading, and therefore the soluble MHC molecules expressed from such cells will not contain peptides bound therein (referred to as free heavy chain HLA). Such soluble, free heavy chain HLA can effectively be tested for epitope binding as well. That is, MHC made in cells which do not naturally load peptide can be experimentally loaded with the peptide of choice. The heavy chain, light chain, peptide trimer can be reassembled in vitro using a high affinity peptide to facilitate assembly. Alternatively, a cell deficient in peptide processing can be pulsed with peptide such that the trimolecular MHC complex forms. DNA encoding a peptide (also encoding an appropriate targeting signal) could also be co-transfected into the cell with the MHC so that the MHC molecule which emerges from the cell is loaded only with the desired peptide. In this way MHC molecules could be loaded with a single low affinity peptide so that replacement with test peptides in a binding assay are more controlled.

Note that an advantage of secreting individual MHC molecules from a cell that naturally loads peptide is that the MHC molecule of interest is naturally loaded with thousands of different peptides. When used in a peptide binding assay, a synthetic peptide can therefore be compared to thousands of naturally loaded peptides.

Thus, the first step of providing a soluble HLA ligand database has been described in detail—i.e. the production of large quantities of sHLA molecules having endogenously loaded peptides. The isolation of the endogenously loaded peptides has also been described such that one of ordinary skill in the art would be capable of producing sHLA molecules and isolating the peptide ligands endogenously loaded therein. Once the peptide ligands have been isolated, the ligands must be individually sequenced, motifs of the ligands must be determined and peptide ligands unique to infected or tumorous cells must be identified, sequenced, and used to generate pooled motifs.

### Epitope Discovery Identification and Sequencing

Once the peptide ligands have been isolated away from the sHLA molecules, they must be: (1) sequenced individually; or (2) sequenced in pools in order to derive motifs of peptide ligands which bind any particular sHLA allele. Additionally, if an infected cell or tumor cell is used as the host, the individual ligands which identify endogenously loaded only in the tumor cell or infected cell (i.e. those peptide ligands which are infected cells and/or tumor cells) must be identified and sequenced individually and/or pool sequenced.

Although class I and class II ligands have been examined by Edman sequencing, the primary characterization of individual ligands has been significantly improved upon through MS/MS applications. This is frequently performed via LC/MS using a microcapillary (<1 mm i.d) HPLC column directly interfaced with a triple quadrupole mass spectrometer. The rationale behind using columns of small i.d. is that a lower solvent flow rate is permitted that ultimately increases the sensitivity of mass spectrometric detection. The methodologies for sequencing individual ligands and pooled ligands are outlined in co-pending U.S. Ser. No. 09/974,366, which has been expressly incorporated herein in its entirety.

Unfortunately, since sample load capacity decreases proportionately to the column diameter, such columns and LC/MS methods can be technically difficult or inconsistent for laboratories not routinely employing them to operate; therefore, more robust protocols for producing and studying class Iderived peptides have been desired. The methodology, as described herein, solves and/or meets this need in the art by a methodology which increases the quantities of ligands extractable by producing recombinant soluble class I and class II molecules. The sample amounts subsequently available offset handling losses at the bench, which have been estimated at 50%, and obviate the need for microcapillary LC/MS prior to MS/MS analysis. This is because significantly larger (20-fold) ligand samples are instead separated by standard offline RP-HPLC followed by NanoES-MS mapping and NanoES-MS/MS sequencing. This methodology has proven consistent for comparatively examining peptides extracted from different class I molecules. Utilizing the production of soluble class I and class II molecules produced according to such a methodology, allows one of ordinary skill in the art to locate and characterize overlapping ligands among distinct allotypes. Additionally, individual ligands may be sequenced. All of this data then is used to provide the sHLA ligand database of the present invention.

In order to develop motifs of all peptides bound by any one specific HLA allele, the purified ligands are fractionated by RP-HPLC. For preliminary diversity assessment, approximately 150 Fg of peptides, as calculated from the ELISA-based total mass of sHLA bound to the affinity column and an estimated 50% handling loss, are loaded in 10% acetic acid onto a 1.0×150 mm C18 column (Michrom Bioresources, Inc.) and separated using an initial gradient of 2-10% buffer B (0.085% TFA in 95% acetonitrile) in 0.02 min followed by a linear gradient of 10-60% buffer B in 60 min at 40 FL/min on a 2.1×150 mm C18 column (Michrom Bioresources, Inc.); buffer A was 0.1% TFA in 2% acetonitrile. Absorbance was monitored at 214 nm, and fractions were automatically collected every minute. For comparative analyses, approximately 400 Fg of peptides were injected in 10% acetic acid containing 2 Fg of the dye methyl violet base B to control for gradient consistency between runs. The gradient formation parameters consisted of 2-10% buffer B in 0.02 min and 10-60% buffer B in 60 min at 180 FL/min. Absorbance was monitored at 214 nm, and fractions were automatically collected every minute. Edman sequencing of fractions, when performed, was conducted on 1/20th of each.

To map extracted peptides and obtain primary sequences, a triple quadrupole mass spectrometer with an ES ion source is employed. By using a triple quadrupole instrument, not only are all of the ions present within a given fraction summarized for a designated mass range (mass mapping), but ions may then be selectively fragmented in order to obtain information from which sequence information can be derived (characterization). This is due to the flexibility afforded by the quadrupole mass analyzers: Q1 and Q3 act as mass filters which can be set to generate alternating DC and RF voltage fields for selectively transmitting specific ions. However, Q2 is an enclosed transmissiononly quadrupole; it can be pressurized with inert gas for the collisional dissociation of an ion transferred through Q1. The specific ionization interface, NanoES, chosen here as an ES source functions on the principles described by developers Wilm and Mann. To establish and validate the procedure, comprehensive peptide mapping and sequencing were first performed among fractions 6 through 19, which represented a region of relatively rich ligand concentration, for B*1501, B*1503, B*1508, and B*1510; once this was accomplished, a more focused, and therefore less extensive, comparison was subsequently made between B*1501 and B*1512.

Prior to NanoES-MS, RP-HPLC fractions are completely dried by speed vac; the peptides were then resuspended in 0.1% acetic acid in 1:1 methanol:water. Aliquots from each of the individually concentrated fractions were loaded into 5 cm gold/palladium alloy-coated borosilicate pulled glass NanoES sample capillaries (Protana A/S). To begin sample flow and data collection, the loaded capillary tube was next carefully opened. The capillary was then positioned directly in front of the API III⁺ (PE SCIEX) triple quadrupole mass spectrometer's orifice, and 20-30 scans were collected as separate data files for the mass range 325-1400 m/z while operating the instrument at positive polarity. This procedure was performed sequentially to obtain constituent mass data for samples drawn from each RP-HPLC fraction.

Spectral ion maps were generated from the TICs acquired for each fraction. The maps obtained from corresponding fractions of peptides eluted from different HLA-B15 molecules were aligned, and ions of interest for NanoES-MS/MS were located. The ion maps were typically compared following baseline subtraction and smoothing. Putative ligand matches or, in the case of B*1512, mismatches among the ions were identified through a combination of data centroiding and direct visual assessment. Preference was placed upon selecting doubly-charged, or [M+2H]²⁺, or higher ion forms commonly resulting from electrospray ionization for subsequent NanoES-MS/MS since the resulting daughter ion spectra were richer than those obtained from the collision of singly-charged, or [M+H]⁺, ions.

NanoES-MS/MS was performed by loading into a NanoES capillary tip, as described above, the desired volume of a fraction for which data was to be acquired. The volume loaded depended upon the relative sample flow rate achieved after opening the capillary tip and how long data acquisition was intended to proceed. Typically 3-4 FL were loaded at a time to collect MS/MS data for 20-25 mid- to low-intensity ions from a given fraction. Once loaded, the source head was positioned and the capillary opened as before. Separate data files were collected for each ion subjected to collisional dissociation.

Daughter ion spectra were generated from the TICs obtained in this manner for each ion chosen. The specific approach taken to interpet individual MS/MS spectra varied from ion to ion depending upon the quality of the data sets obtained but adhered to the general rules of MS/MS fragment interpretation. The Predict Sequence algorithm included as part of the BioMultiView software (BioToolBox package, PE SCIEX) was employed for the de novo sequence interpretation, and the sequences deduced were checked for identity with source proteins in various databases using the PeptideSearch algorithm and performing advanced BLAST searches against the National Center for Biotechnology Information (National Institutes of Health) databases.

Leu and Ile were indistinguishable unless suggested by Edman data and/or specific sequence matches, as were Gln and Lys since lysyl derivatization prior to fragmentation was not performed. NanoES-MS/MS data from ions of potentially overlapping peptides was aligned to confirm or refute the presence of shared ligands among different HLA-B15 molecules, as shown for one ion confirmed as an overlapping peptide across B*1501, B*1503, and B*1508. To establish a numerical description (Nₛᵤₘ/Cₛᵤₘ) comparing ligand—and C-regional occupancies for each allotype, N and C values for the four ligand positions at either terminus were determined by summing occurrence frequencies (using an arbitrarily-defined baseline of 10%); Nₛᵤₘ was subsequently calculated from the four N values, and Cₛᵤₘ was calculated from the four C values.

Peptides from HLA-B15 molecules were subjected to pooled Edman sequencing as well as more extensive examinations, including fractional Edman sequencing and mass spectrometric characterization of individual ligands. This was done to: (i) confirm the production/purification methods employed; and (ii) evaluate the relative nature and complexity of the peptides contained in extracts of naturally presented ligands.

Upon extracting peptides from each of six different B15 molecules, pooled Edman sequencing was performed. This was done both to validate results from the extraction of sHLA ligands with the techniques previously employed by others, and to obtain novel motifs from the molecules that had not been previously examined (B*1503, B*1510, B*1512, and B*1518) for providing “traditional” points of reference.

Overall, the B*1501 motif was found to be in agreement with the dominant P2 and P9 anchors (Gln and Tyr/Phe, respectively) previously defined. This result demonstrates that the peptides extracted from sHLA-B*1501 complexes were identical to those extracted by others from natural membrane-bound molecules. However, differences in the whole pool sequencing data arising from sHLA purified by BBM.1 versus W6/32 indicated that a greater number of peptides than originally realized should actually contribute to the consensus B*1501 motif.

Indeed, without the ability to generate motifs based upon 1,000s of peptide sequences (as opposed to typical 10-150 sequences) some if not all of these additional sequences of peptides bound might be missed. As has been shown in the art, as low as 20 peptides may produce an immune response. The greater the number of sequences used to determine motifs, the more viable the motifs become for use as screening and prediction tools.

First, while Gln and other residues including Leu, Met, and Val have been previously reported at P2 using W6/32 to isolate complexes, the aliphatic side chain Pro was strongly detected at P2 in the BBM.1-purified B*1501 motif as well. Though not employed by other groups pursuing similar studies, the β₂m-specific MAb BBM.1 was initially chosen here to avoid biases potentially imposed upon the class I heavy chain by bound peptides. Of interest was that Pro has not been reported before as a strong or even weak P2 anchor in the B*1501 peptide motif. It has been suggested that B-locus allotypes that present peptides with Pro at P2 demonstrate a shallower B-pocket within their binding grooves than does B*1501, which exhibits a Ser at α-chain position 67 rather than a more constricting residue such as Phe.

This data suggests the possibility that Pro binds amicably within this deeper pocket but perhaps induces an altered heavy chain conformation that negatively biases purification of complexes by the W6/32 MAb typically used. The observation of a P2 Pro occupying a pocket with suboptimal physical complementarity is corroborated by a similar occurrence among peptides bound by the murine class I molecule L^{d}. Purification methodology serves, therefore, as a factor in allele-specific motif predictions, and the whole pool sequencing with peptides extracted from both BBM.1 and W6/32-purified B*1501 complexes demonstrated that a strong Pro anchor at P2 is antibody dependent.

The pooled Edman motifs obtained for W6/32-purified molecules divergent from B*1501 are shown and tabulated in co-pending application U.S. Ser. No. 09/974,366. Like B*1501, each of the motifs reflected a nonameric consensus with distinct P2 and P9 anchors and internal auxiliary anchor preferences. The B*1508 motif, described by another group while its preparation was in progress during the development of this methodology, was consistent between the two laboratories; it demonstrated a preference for the small side chains Pro and Ala at P2 and aromatic residues Tyr and Phe at P9. The B*1512 motif appeared nearly identical to that obtained from B*1501; by extension, considering that B*1519 differs from B*1512 in α₃, which does not contribute to the peptide binding groove, it is predicted that B*1519 would bear the same motif as B*1501 and B*1512.

B*1503 diverges somewhat from the other three molecules presented above in showing a distinct preference for ligands with a neutral, polar Gln or positively-charged Lys as the P2 anchor; the aliphatic Met was evident here as well, though to a lesser degree than noted for the hydrophilic Gln and Lys residues. Like B*1501, B*1508, and B*1512 however, aromatic residues Tyr and Phe defined a hydrophobic P9 anchor. The only other class I molecules with motifs whose definitions thus far indicate a Lys at P2 are B*3902 and B*4801, both of which structurally bear B-pockets identical to B*1503 except for a single L6T or L6E substitution, respectively, at the α2 helical residue 163. The B-pocket of B*1503 is indistinguishable from that of B*4802, whose motif remains undetermined but is likely to follow suit with those of B*1503 and these other molecules at the second ligand position. An assortment of polar, charged, and hydrophobic residues is evident at P3 of the B*1503 motif.

The B*1510 motif demonstrated a strict preference for ligands bearing a basic, hydrophilic His as a P2 anchor. A hydrophobic P9 anchor was described by residues including Leu and Phe. The B*1510 motif strongly resembled that previously defined for B*1509, which exhibits nearly identical anchor preferences with His at P2 and Leu, Phe, and Met at P9. B*1510 and B*1509 differ structurally only by a substitution of N6D in α₂ at the α-sheet floor position 114, which takes part in forming several specificity pockets within the peptide binding groove.

By extrapolation from its structural neighbors, it was assumed that B*1518 would have for its motif a P2 anchor of His (as seen for B*1510 and B*1509) and a P9 of Tyr and Phe (as seen with B*1501, B*1503, B*1508, and B*1512). B*1518 differs from B*1510 solely at position 116; two other HLA-B molecules that differ exclusively at this position are B*3501 and B*3503: they differ by a S6F substitution here, which would sterically mimic the substitution between B*1518 and B*1510 and confer B*1510-like P9 preferences (Steinle et al. 1995; Kubo et al. 1998). Based upon this, and the fact that the P9 environments of B*1518/B*3501 and B*1510/B*3503 are similar, it was first predicted, and then confirmed following pooled sequencing, that B*1518 would bear the hybrid motif described.

Pooled Edman sequencing data therefore demonstrates that (i) the peptides extracted from sHLA complexes produced according to the methodology described herein are consistent with those previously extracted from native, cell surface-expressed complexes, and (ii) nonameric ligand lengths with anchor residues at P2 and P9 characteristic to specific polymorphisms are preferred. As for functional implications, the major anchors would predict natural ligand overlaps with B*1501 by B*1503 and B*1512. Thus, combined with the large scale production of sHLA, peptide ligand sequences and pooled motif sequences are readily discernable in a standardized and normalized manner for any and all HLA molecules. Such standardized and normalized data, when populated in a searchable and robust database structure provide a unique and highly valuable research tool for vaccine development.

Since class I peptide pools consist of thousands of different ligands, methods were developed to next fractionate and then Edman sequence the peptides extracted from one of the molecules as an example of its capability for all sHLA alleles. BBM.1-purified B*1501, the first soluble molecule produced by a non-repeatable precursor methodology to the fully repeatable and characterized methodology of the present invention, was initially examined to explore the general diversity around a pooled motif.

Edman sequencing of peptide-containing fractions collected from the RP-HPLC gradient supported the existence of peptides up to 12 residues long and revealed significant positional diversity among the peptides isolated from B*1501. This positional diversity was illustrated in 16 representative fractions. Assessment of dominant, strong, or weak amino acid residues present at the various positions indicated that the dominant anchors previously defined by whole pool sequencing did not necessarily predominate in fractions of the peptide pool. In fractions 15 and 31 the dominant P2 Gln was replaced by a dominant Ala and a dominant Lys respectively, while the pooled motif Tyr fell below residues such as His and Lys at P9 in the same fractions. While variations on the consensus motif were prevalent at P2, this was not solely restricted to the N termini of bound peptides.

Among the 16 fractions studied, 12 demonstrated weak sequence yields out to 12 cycles of degradation; in nine of these fractions, P12 was occupied by the charged or polar residues Glu, Arg, Lys, Ser, His, or Gln. Additionally, the presence of numerous decamers within the B*1501 peptide population was suggested in that 11 of the fractions sequenced exhibited a typical P9 residue, Tyr or Phe, at P10. For example, the strong Phe presence at P10 in fraction 28 suggests that P10 serves as an anchor for a decamer(s) present within the fraction. The shifting of a P9 anchor preference to P10 is consistent with the P10 occupancies of individual decamers formerly characterized from B*1501 peptide pools. Although residues representing P9 anchors were seen at P10 and P12, amino acids unique in such longer ligands were also detected, suggesting that the shifting of a previously-reported P9 anchor is not the only means by which longer B*1501 peptides are bound within the peptide binding groove.

During the course of examining ligands from other sHLA molecules, which were purified alternatively with MAb W6/32, aliquots of RP-HPLC fractions from some were also subjected to Edman sequencing on occasion. The results from these random samplings of B*1501, B*1508, B*1503, and B*1510 fractions are summarized for major characteristics in co-pending application U.S. Ser. No. 09/974,366. The characteristics recorded included preferences other than those seen in the pooled motifs at traditional anchor positions P2 and P9 and whether signal levels indicated the presence or not of residues beyond the nine cycles of degradation typically observed. Of additional interest, no Lys was observed among Edman sequenced fractions as an additional preference at P2 for B*1501 purified with W6/32, a finding contrary to the P2 preferences among fractions of BBM.1-purified material which lends further support to the argument for MAb bias, as discussed earlier, existing in complex purification.

In summary, the fractionation of B*1501 peptides priorto Edman analysis resulted in amino acid sequence data demonstrating that the components of a peptide pool can vary considerably from the overall motif. These data, as well as results obtained from other B15 molecules, suggested that peptides bound by the various molecules include: (i) species which are either longer or shorter than the nonameric size typically indicated by pooled sequencing alone; and (ii) species that exhibit primary sequences different from those predicted by pooled sequencing. The additional data provided by further exploration of fractionated extracts from several of the B15 molecules in this manner expands the molecules with the potential for presenting B*1501-overlapping ligands to include not only B*1503 and B*1512 but B*1508 and B*1510 as well. Such diversity among fractions indicates that characterization of individual ligands would provide information not available in motifs.

Individual peptides from B*1501, B*1503, B*1508, B*1510, and B*1512 were comparatively examined to investigate whether the added flexibility observed through Edman degradation of RP-HPLC fractions would allow for natural ligand overlaps to occur across their respective polymorphisms. More than 400 individual ligands extracted from the five distinct HLA-B15 allotypes were characterized according to the present methodology. The ligands characterized here were from ion map masses found in multiple B15 allotypes. Selected ions were then dissociated by NanoES-MS/MS, and the resulting fragment information was compared and interpreted, as described hereinabove, to determine if the ions represented sequence-identical or merely mass-identical ligand matches.

Individual peptide ligands characterized from the five B15 allotypes are listed in Tables A-E of co-pending application U.S. Ser. No. 10/022,066. The number of ligands for which either complete or partial sequences were obtained here was as follows: B*1501=126, B*1503=74, B*1508=96, B*1510=123, and B*1512=30. While the pooled motifs of peptides extracted respectively from the five molecules described nonamers with various P2 and P9 dominant anchors and P3 auxiliary anchor preferences, the single peptide sequences ranged from 7 to 12 amino acids in length and demonstrated (i) greater heterogeneity at their N-terminal/proximal regions than their C termini, and (ii) varying degrees of observed ligand overlap, both of which will be examined in the subsequent sections of this chapter.

In terms of length heterogeneity, the endogenous peptides eluted from B*1501, B*1503, B*1508, B*1510, and B*1512 varied in length from 7 to 12 amino acids. An overall length breakdown of the peptides listed in Tables A-E of co-pending application U.S. Ser. No. 10/022,066 demonstrates that approximately 6% are heptamers, 21% are octamers, 50% are nonamers, 19% are decamers, 3% are undecamers, and 1% are dodecamers. Further emerging from the length characterization of individual ligands is the observation that peptides bound by each of the B15 molecules spanned ranges of 5 to 6 amino acids in length. For example, peptides eluted from B*1501, B*1510, and B*1512 were 7-11 amino acids in length, while those from B*1503 and B*1508 were 7-12 amino acids in length.

Coupling this length variability with the likewise varying degrees of regional sequence heterogeneity (which will be discussed) leaves only 23% of the endogenously loaded peptides characterized as “ideal nonamers” with both P2 and P9 anchors in concordance with the dominant or strong preferences of the pooled Edman motifs from their respective source molecules. This finding is of principal significance in that a majority (77%) of potential ligands for any of these HLA-B15 molecules would therefore be overlooked if the length and sequence constraints of their pooled motifs were utilized as the primary criteria in searching for potential epitopes specific to them.

Examples of ligands from this study with homology to stretches of known proteins are shown in Table 4 of co-pending application U.S. Ser. No. 09/974,366. The peptides yielding 100% identical BLAST database hits were grouped into seven categories, which were defined according to the common natures of their potential source proteins: HLA ligands, replication/transcription/translation ligands, biosynthetic/degradative modification ligands, signalling/modulatory ligands, transporter/chaperone ligands, structural/cytokinesis ligands, and unknown function ligands. Aside from the HLA heavy chain-derived ligands, most appear to be derived from cytoplasmic or nuclear proteins, which illustrates that the typical endogenous pathway is involved in generating the majority of the class I-loaded peptides characterized.

Of the 44 peptide sequences listed in Table 4 of co-pending application U.S. Ser. No. 10/022,066, it is noteworthy that overlaps across other HLA-B15 molecules are evident within our data collection. The B*1510 tapasin₃₅₄₋₃₆₂ ligand HHSDGSVSL, as well as both THTQPGVQL from septin 2 homolog₇₀₋₇₈ and SHANSAWL from α-adaptin₂₄₉₋₂₅₇, have also been sequenced from B*1509 extracts, and the B*1501/B*1508/B*1512 ubiquitin-protein ligase₈₃₋₉₁-derived ligand ILGPPGSVY was characterized from endogenously bound B*1502 peptides. The eIF3-p66₆₁₋₆₉ nonamer SQFGGGSQY was found here within B*1501, B*1503, B*1508, and B*1512 extracts. The decamer YMIDPSGVSY, which is homologous to proteasome subunit C8₁₅₀₋₁₅₉, was also previously described as a ligand for B*1502, B*1508, and B*4601; it was found here presented by B*1501, B*1508, and B*1512. Some of the specific overlapping ligands identified in this study therefore overlap in antigen presentation with the HLA-B15 allotypes characterized by others.

Given the length heterogeneity observed among the ligands collectively characterized from B*1501, B*1503, B*1508, B*1510, and B*1512, analysis of peptide ligand primary structures proceeds through two separate alignments (N- and C-terminal) for each HLA-B15 allotype. The frequencies with which specific side chains occurred at (i) the N terminus and first three residues internal from it, and (ii) the C terminus and the first three residues internal to it were tabulated. The salient features of these ligand regions and how they correlated with the structures of the molecules from which they were extracted can be examined.

The N-terminal/proximal regions for ligands from each of B*1501, B*1503, B*1508, B*1510, and B*1512 clearly demonstrated acceptance of a variety of amino acid side chains, particularly at P3 and P4, by the portions of the binding groove assumed to interact with ligands at the designated positions. With the exception of B*1512 ligands, which were obtained from both a smaller and more biased collection of ions, higher points in each of the graphs occur for certain side chains indicated along the P1 and, to a greater extent, P2 data lines, which represent the first and second positions, respectively, of the characterized ligands.

The results for P1 obtained from the HLA-B15 ligands characterized are of interest since the analysis of Edman sequencing data depends upon comparing relative increases between cycles and is therefore unreliable for making side chain determinations at this first position, especially when complex mixtures of peptides are examined. All five B15 allotypes demonstrated a number of side chains at P1, with preferences for residues including Ala, Leu/Ile, Gly, Ser, Thr, and Tyr observed in varying degrees among them. Overall, P1 appeared to be occupied in a majority of ligands by aliphatic amino acids.

Though the pooled motifs of B*1501 and B*1512 as well as the spectral ion maps obtained from their RP-HPLC fractions were virtually identical, the substitution difference between the two molecules at A-pocket residue 167 suggested that ligands bound by these molecules might differ at P1. Performing NanoES-MS/MS upon a handful of ions, which appeared to be exclusive to B*1512, confirmed the presence of several ligands presented by B*1512 but not B*1501. Of the 16 sequenced, seven indicated His, two indicated Arg, and one indicated Lys at P1. In comparison, only a single B*1501 peptide each presented with His, Arg, or Lys at P1 out of 126 ligands characterized. An explanation for the existence of this subset of B*1512-restricted ligands with positively-charged N-termini could lie in the W6G substitution observed between B*1501 and B*1512 at α₂ position 167, which might sterically enhance the influence by the adjacent acidic residue at position 166 (Glu in B*1501 and Asp in B*1512) of B*1512 upon P1 in the A-pocket. Comparing individual ligands between B*1512 and B*1501 supports the notion that the polymorphism segregating them will confer distinct yet subtle effects upon peptide binding by other allotypes differing in this manner.

P2, which has been considered to act as a primary anchor for peptide ligands among most class I molecules described to date as based upon pooled Edman motifs, is classically accepted to associate with the B-pocket of the peptide binding groove. In terms of the motifs derived by pooled sequencing and the motifs previously established for other B15 family allotypes, a GIn at P2 is common to B*1501, B*1502, B*1503, B*1512, and B*1513. Three alleles, B*1502, B*1513, and B*1508, have a Pro at P2, while the lack of a strong Pro at P2 in both B*1501 and B*1503 corresponds to polymorphism at heavy chain positions 63 and 67. For example, B*1501 appears to lose the propensity for Pro at P2 due to polymorphism at position 63, while B*1508 appears to lose a Gln at P2 resulting from polymorphism at 67. Thus, comparisons within the B15 family highlight how substitutions at positions 63 and 67 of the class I heavy chain α₁ helix appear to confer differential interaction with P2 of the peptide ligand.

While residue 63 modulates the size/conformation of P2, it can be seen that residues 24 and 45 influence the P2 charge nature propensities. A comparison of B*1501 and B*1503 illustrates how polymorphisms at positions 24 and 45 of the B-pocket influence P2 preferences in this manner; B*1503 is one of four B15 alleles with known motifs bearing a positively charged P2. Allotypes B*1509, B*1510, and B*1518 recognize a positively charged His at P2 and have the same residues at 24 and 45 as B*1503, but the differences at positions 63 and 67, which separate B*1503 from the other three molecules, again modulate the contour of P2 such that different positively charged P2 residues fit respectively into the B*1503 and B*1509/B*1510/B*1518 B-pocket categories.

It has previously been proposed that polymorphisms in the α₁ helix prompt major changes in the repertoires of peptides bound by allotypes differing in this region. That any region, helical or sheet, of α₁ would influence peptide P2 preferences more than α₂ is of little surprise though since 14 of the 18 residues forming the B-pocket belong to the α₁ domain. A comparison of 12 known B15 motifs in the B-pocket suggests more refined rules for α₁ in general, whereby polymorphisms in the helices sculpt the conformation and size of the amino acids that can fit into the peptide binding groove's B-pocket. Further analysis of the B15 motifs at P2 suggests that polymorphisms lining the floor of the groove tend to regulate the hydrophobic and/or charged nature of the residues at P2 of bound ligands. Perhaps in this way the walls and floor of the binding groove work in concert: the α-helical residues sterically control which amino acids can fit, while the α-sheet residues act to attract or repel particular side chains based on chemical compatibility within the ligand binding groove.

The interactions described for P2 here are, however, more relaxed than previously thought. For a majority of the allotypes known, three or more different side chains are observed by pooled Edman sequencing as dominant or strong B-pocket residents and/or the B-pockets of the molecules demonstrate abilities to naturally accommodate alternative P2 residues. Of specific interest, although Met appears in the B*1503 pooled motif as a strong P2 anchor residue, only two of the peptides characterized for this allotype bear Met at P2, while other residues including Gly, Pro, Ala, and Asn occur more often than Met at P2 among B*1503 ligands. The fact that Met appears in the pooled motif but fails to demonstrate a strong presence among the individual peptides indicates that disparate concentrations of ligands within extracts may skew pooled Edman sequencing results so as to be misleading.

Pro and Ala likewise appear with frequencies comparable to or exceeding those of the W6/32-purified pooled motif residues for B*1501, and B*1508 ligands illustrate P2 inclinations for a rich array of side chains in addition to the motif-prescribed residues Pro and Ala which include Gly, Val, Met, Leu/Ile, Ser, Thr, and Gln/Lys. Similar variety is observed within the limited B*1512 ligand data set. In contrast, the B-pocket composition for B*1510 indicates His as the sole dominant/strong P2 occupant, and among individual ligands characterized from B*1510 His is noted at a markedly higher degree than are alternative residues. However, amino acids including not only the positively charged Arg but to a greater extent Gly, Ala, Val, Leu/Ile, and Gln/Lys occur at P2 of some peptides are also characterized at P2 from this allotype. Thus the majority of HLA-B15 molecules demonstrate elastic N-proximal occupancies. In comparison with the findings at the N-terminal/proximal regions, the C termini of ligands from each of B*1501, B*1503, B*1508, B*1510, and B*1512 demonstrated a stricter acceptance of amino acid side chains. C-proximal ligand residues also revealed the existence of more distinct side chain tendencies.

For allotypes B*1501, B*1503, B*1508, and B*1512 a dominant C terminus was especially prominent among the ligands characterized from them, while B*1510 exhibited a P2 anchor nearly as strong as its primary C-terminal residue preference. The aromatic residues Phe and, even more prominently, Tyr occupied the C-terminal positions of most peptides bound by the first four B15 molecules, which appeared to agree with P9 of their respective motifs. The bulk of B*1510 ligands demonstrated Leu/Ile at their C termini; other occupants at this position included Phe and Val, an interesting observation in that more B*1510 peptides presented with Val, which is not included in either the pooled or fractional Edman motifs examined from this allotype, than Phe, which is identified as a strong P9 occupant by pooled sequencing. Such is the likely result of disparate peptide concentrations affecting the pooled Edman sequencing results as mentioned previously. Another factor includes the diminishing picomole yields per successive cycle of Edman degradation; this leads to progressively higher background signals and thus negatively affects sensitivity in examining the C-terminal/proximal regions of peptides.

The overwhelming conservation at the C termini of individual ligands indicates that the C terminus acts as a dominant anchor for peptide ligands. P9 of pooled Edman motifs has classically been accepted to associate with the F-pocket of the peptide binding groove. C-terminal anchoring is observed here regardless of length heterogeneity. For the majority (91%) of peptides greater than 9 residues long, this observation agrees with evidence that longer peptide ligands bulge centrally outward from the peptide binding groove. Sequencing individual ligands supports a concept that the C terminus of a ligand plays a dominant role as an anchor within the class I binding groove for the HLA-B15 allotypes examined.

With regard to pooled sequence motifs for HLA-B15 allotypes, all 12 molecules demonstrate chemical homogeneity at P9, with dominant/strong occupancies by hydrophobic residues. The eight different F-pockets structurally represented among these allotypes show preferences for Tyr, Phe, Met, Leu, and Trp according to three functional group categories. This is in marked contrast with the B-pockets, for which eight different B-pockets among the same allotypes comprised seven distinct functional groups encompassing a mixture of both hydrophobic and hydrophilic side chains.

It is interesting that, of these HLA-B15 molecules, nine have F-pocket functionality in the same category (B*1501, B*1502, B*1503, B*1508, B*1512, B*1516, B*1517, B*1518, and B*4601), with preferences for Tyr, Phe, and/or Met, despite the fact that they exhibit amino acid substitutions at nine different positions throughout the α₁ helix and β₂ sheets. This redundancy demonstrates that, contrary to what was seen among structural residues affecting the B-pocket, the α₁ helical polymorphism(s) shown for allotypes do not necessarily play a defined role in sculpting either the conformation or size preferences of ligands in this region of the peptide binding groove.

While the Edman-derived motifs for the B15 allotypes clearly indicated P2 and P9 primary anchors and suggest an assortment of preferences at both P3 and P4, they fail to sufficiently capture the trends for auxiliary anchoring at the C-proximal regions of endogenously bound ligands which were perceptible throughout the individual peptide sequences. Additional preferences that likely serve as auxiliary anchors were evident at the C-proximal positions C⁻¹, C⁻², and C⁻³ in the cases of nonamers, octamers, and heptamers, as well as in ligands longer than nonamers. A review of the positional frequencies observed among the HLA-B15 peptides shows that amino acids such as Val, Leu/Ile, Ser, Thr, and Gln/Lys tended to predominate at nearly all three of the C-proximal positions of ligands presented by B*1501, B*1503, B*1508, B*1510, and B*1512. Of these residues, the hydrophilic, hydroxyl-containing Ser and Thr were especially frequent among these positions; nearly half (40%) of the ligands listed in Tables A-E of co-pending application U.S. Ser. No. 10/022,066, have Ser and/or Thr at the designated C-proximal positions. In general, Glu occupied C⁻¹ and Gly occupied C⁻³ to some extent among all five allotypes.

Val (C⁻¹) and Pro (C⁻²) were especially prominent C-proximal residues observed among the B*1510 ligands; the overriding presence of Pro, which distinguished this region of B*1510-derived peptides from those of the other allotypes, can likely be attributed to steric influences imposed by the Tyr at α₂ position 116 in B*1510, which additionally interacts with the C- and E-pockets of the peptide binding groove. Further distinguishing several B*1510 ligands from but rare occurrences among B*1501, B*1503, B*1508, and B*1512, Pro frequently appeared as well in various C-proximal sequence combinations with Ala or Val.

The amino acid residues characterized from each of the five HLA-B15 allotypes with occupancy rates of at least 10% for the first four (N-terminal/proximal) and last four (C-terminal/proximal) positions among ligands, respectively, are condensed in Tables 9-13 of co-pending application U.S. Serial No. 10/022,066. Presenting the data already discussed in this manner effectively emphasizes C-terminal dominance and N-proximal flexibility. By comparison, the data illustrates the limitations of pooled Edman motifs in being able to adequately reflect a consensus of the individual peptides contained within a given ligand extract. The Nₛᵤₘ/Cₛᵤₘ quotients obtained as described were less than 1.00 in the cases of all allotypes, thus providing a more fixed description to the C-terminal/proximal region (gray) as a whole with respect to the N-terminal/proximal region (black).

Among the N-regional position ligand residues occurring at >10%, nothing appears to prominently stand out at P3 and P4 although assignments were made to these positions via Edman sequencing. The occupancies that were observed were not necessarily captured by the motifs; a specific illustration of this is Ala (19.51%) at P3 of B*1510. This trend appeared likewise applicable at the P2 anchor, where with the exceptions of B*1508 and B*1510, occupants at this position among >10% of ligands from each of the remaining allotypes included additional side chains (for example, Ala at P2 in both B*1501 and B*1512) not accounted for by pooled sequencing. The C termini of each allotype are comprised of two amino acid specificities as shown by more than 80% of characterized peptides in all cases. In summary, comparing observed—and C-regional occupancies among the characterized ligands underscores the flexibility of N-proximal versus the dominance of C-terminal preferences among the B*1501, B*1503, B*1508, B*1510, and B*1512 binding grooves.

A total of 40 specific ligands among the 449 characterized here (Tables A-E) (found in copending U.S. Ser. No. 10/022,066) overlapped across the peptide binding grooves of B*1501, B*1503, B*1508, and/or B*1512; as previously discussed from the information in Table 4 (found in copending U.S. Ser. No. 10/022,066), some of the overlapping ligands likewise coincided with ligands characterized by others from additional HLA-B15 allotypes including B*1502 and B*4601. Length variations among the overlapping ligands identified tended to mimic those observed among the entire set of ligands characterized. Only seven overlapping ligands were longer than 9 amino acids in length (4 decamers and 3 undecamers), while 16 fell short of 9 residues long (6 heptamers and 10 octamers); less than 50% of the successful overlaps were therefore nonameric.

Throughout the mapping and sequencing approach that was developed and executed as outlined earlier above, an extensive comparison was first conducted upon ions occupying RP-HPLC fractions 6-19 from separations of ˜400 Fg of peptides from each of B*1501, B*1503, B*1508, and B*1510, the first four B15 molecules prepared in the course of this study. From this, 21 peptide overlaps across B*1508 and B*1501 were defined. Similarly, eight ligands overlapping B*1501 and B*1503 were identified, and four ligands were found to overlap across B*1508, B*1503, and B*1501. A conservative estimate, based upon past examination of B*1501 ligands, is that the ion maps for each of the B15 allotypes represented at least 2,000 individual peptides per molecule, yet B*1510 was not observed to share ligand overlaps with B*1508, B*15011, or B*1503.

The sequence data indicates that overlapping ligands bind across divergent B*1508, B*1501, and B*1503 binding grooves but not B*1510. This pattern likewise accentuates an apparently dominant role for C-terminal anchors in natural peptide binding as discussed previously. In the context of the class I peptide binding cleft, the locations of polymorphisms that individuate B*1508, B*1501, B*1503, and B*1510 and highlights the anchoring residues for the peptide overlaps according to the N-proximal and C-terminal specificities of their respective presenting molecule's motif. Bolding the amino acids of these overlapping ligands, which are in agreement with the traditional pooled motifs, underscores the trend whereby a C-terminal anchor sequence is conserved within overlaps while the N-proximal anchor is considerably more flexible in its location and/or sequence. A lack of overlaps with B*1510 could potentially be explained by the S6Y substitution observed between this allotype and the other three α₂ position 116. Thus, the conserved C-terminal anchors that facilitate the occurrence of B*1508, B*1501, and B*1503 overlaps fail to preferentially interact with the B*1510 C-terminal specificity pockets.

A further example provided here of how C-proximal auxiliary anchors might positively impact endogenous ligand binding is that eight of the peptides overlapping both the B*1508 and B*1501 antigen binding grooves bear Thr at C₋₁, C⁻², or C⁻³, and in four cases the peptides that bind B*1508/B*1501 or B*1508/B*1501/B*1503 are heptamers with Thr occupying P7, their C-terminal positions. The prominent role of Thr as a C-terminal/proximal auxiliary anchor is dramatically illustrated by the B*1508/B*1501/B*1503 overlapping heptamer CPLSCFT, where Thr provides a C-terminal anchor for this ligand not evident in the pooled motifs of the three allotypes.

Distilling the data from the overlapping ligands among B*1501, B*1503, and B*1508 suggests a model for endogenous ligand binding whereby peptides are first anchored or held in the class I binding groove by their C termini. In order for a given peptide to remain stably fastened in the groove for successful trimer assembly and subsequent export from the cell, it is observed that following rigid anchoring at the C terminus as described, a ligand must be subsequently tethered into the class I antigen binding cleft at a more variably defined N-proximal position. Such is the case for peptide ligand NQZHGSAEY, a nonamer that overlaps across B*1508, B*1501, and B*1503. According to this model, a C-terminal Tyr securely anchors NQZHGSAEY into all three B15 allotypes, while a Gln at P2 anchors the peptide into B*1501 and B*1503 and a Gln/Lys (most likely a Lys based upon both motif assignments and fractional Edman sequencing data) at P3 provides additional anchoring for B*1501 and serves as the sole N-proximal anchor for B*1508. This model appears clearly applicable to at least 75% of the ligands presented in FIG. 26; for those peptides to which it does not evidently apply, the possible anchoring modes remain open to further speculation at the level of individual ligands.

For example, the B*1501/B*1503 overlap AQFASGAGZ may instead be additively stabilized through the N-proximal anchors indicated at P2 and P3 as well as at the N-terminal position, since Ala demonstrated significant P1 occupancy among both B*1501 and B*1503 ligands, as previously shown. Additionally, the four heptameric overlaps that were observed across B*1508/B*1501/B*1503, which terminate in Thr, could lie within the peptide binding groove such that they are anchored N-terminally/proximally and their C termini interact with the C-proximal regions of the groove, which have demonstrated preferences for Thr; these ligands might therefore fail to extend into the F-pocket. As compared with C-terminal sequences, both length and N-proximal specificity characteristics of ligands generally play secondary roles in the natural binding of B15 peptide epitopes.

Further stemming from the data obtained by comparatively examining B*1501, B*1503, B*1508, and B*1510 ligands, differential interactions with the chaperone tapasin specifically influence the loading of peptides into HLA-B15 molecules. Tapasin, an MHC-encoded chaperone discussed hereinabove, is a recently-discovered 48 kDa transmembrane glycoprotein resident to the ER that directly interacts with both calreticulin and empty α-chain/β₂ m dimers to form a “loading complex” linked to TAP1/TAP2. Tapasin is not a requirement for ligand loading via the typical endogenous processing pathway, and aside from its proposed role in serving as a bridge between a class I dimer and the peptide transporter until release of mature trimers upon peptide binding, the exact role of tapasin during class I assembly is unknown. Interactions between nascent class I molecules and TAP1/TAP2 have, however, been shown to be influenced either directly or indirectly by α₃ and positions 116 and 156 of α₂.

Of specific interest, past analysis of divergent HLA-B35 molecules has indicated that allotypes bearing an aromatic amino acid (Phe orTyr) at position 116 interacted with TAP1/TAP2; allotypes bearing a Ser substitution at this site failed to demonstrate the interaction. Likewise, data subsequently obtained for the sHLA transfectants utilized here according to established immunoprecipitation protocols indicates that although all four allotypes associate with calreticulin, B*1501, B*1503, and B*1508 do not associate with tapasin (and therefore not with TAP1/TAP2) whereas B*1510 does. Membrane-bound forms of B*1501 and B*1516 have previously been shown by others to not associate with TAP1/TAP2, demonstrating that results obtained from the sHLA transfects are in concordance with those of native molecules.

Though functionally divergent according to its pooled motif and the majority of peptides that it binds, B*1510 is capable of accommodating ligands with the properties favored by the B*1501, B*1503, and B*1508 binding grooves. Data both from individual ligands and fractional Edman sequencing indicate that Tyr can occupy the C-terminal position, including the spleen mitotic checkpoint BUB3₅₃₋₆₀ octamer YQHTGAVL and the splicing factor U2AF large chain₁₇₉₋₁₈₇ nonamer TQAPGNPVL, attest to B-pocket flexibility. It is intriguing that among the peptides bound by B*1510 is the tapasin₃₅₄₋₃₆₂ nonamer HHSDGSVSL; the peptide appears to occupy ligand extracts in a high copy number, as qualitatively based upon relative mass spectrometric ion intensities. Given this, as well as considering potential models of loading complex interactions suggested by others, it can be extrapolated that a portion of tapasin, analogous to class II-associated invariant chain-derived peptides, extends into and blocks a region of the empty class I binding groove until it is displaced by an optimally-fitting ligand and/or secondary chaperone; this could also account for the differences in overall P2 flexibility observed between B*1510 peptides and those of the other three allotypes. Participating in ligand selection by this mechanism would describe a distinct peptide editing role for tapasin and could clarify the inability to detect overlaps between B*1510 and either B*1501, B*1503, or B*1508.

In addition to the initial search for overlaps across B*1501, B*1503, B*1508, and B*1510, a comparative analysis was performed between the ion maps of B*1501 and B*1512. As discussed previously hereinabove, such an examination is primarily important in revealing the presence of ligands bound by B*1512 but not B*1501. A number of overlapping ligands from B*1512, however, were additionally identified. Conservative percentages of overlap subsequently observed among each of the four molecules from which ligands were characterized and the ancestral HLA-B15 allotype, B*1501, were determined.

As expected from an overview of their nearly identical ion maps, B*1501 and B*1512 demonstrated the highest overlap frequency between the allotypes at 70% among ions subjected to NanoES-MS/MS. After this, B*1503 and B*1508 respectively exhibited 14% and 9% overlap frequencies, while as shown earlier B*1510 completely failed to reveal overlaps with B*1501. The trend distinctly illustrates that the polymorphisms which distinguish the B*1503, B*1508, B*1510, and B*1512 peptide binding grooves from B*1501 are not functionally equivalent in terms of their impacts upon class I ligand association. However, it is also evident that they do not create concrete barriers to ligand binding. Because the repertoires of peptides bound by the various molecules examined may differ from B*1501 at frequencies greater than 80% (B*1508 and B*1503) does not mean that they are unable to bind similar or completely identical peptides, a concept which has been incompletely addressed and occasionally negated by other studies grounded more upon pooled Edman sequencing, analysis of prominent extract constituents, or binding/reconstitution assays.

In particular and based upon previous research, the overlaps between B*1508 and B*1501 defined here would specifically not have been predicted; it may be anticipated by extension that other molecules differing solely by the polymorphism separating B*1508 and B*1501, for example B*1503 and B*1529, yield similar overlap frequencies. Likewise, based upon the relatively high overlap frequency observed between B*1512 and B*1501, the substitutions at positions 166 and 167 distinguishing them exhibit a similarly subtle effect between A*2902 and A*2903 (which only differ from one another by the identical substitutions studied here;) via mapping and sequencing of individual ligands. Systematically attempting to define the limits of overlap existence as conducted, therefore, demonstrates a critical departure from standard approaches which enhance predicting the abilities of different class I molecules to present overlapping ligands.

Comparative analyses of closely related soluble MHC class I molecules produced by the recombinant methods described herein, provide a means for assessing the functional impact of individual α-chain polymorphisms. The primary impetus for characterizing peptides extracted from class I molecules is to more precisely understand the influence of structural polymorphism upon the presentation of endogenous ligands. This is important since a fundamental realization of how naturally processed peptides bind to both individual and multiple class I allotypes can then be translated into protein and/or peptide-based therapies intended to elicit protective CTL responses. Therefore, an accurate interpretation of sequence data from such class I-bound peptides, either individual or pooled, should in turn further the selection of optimal viral and tumor-associated ligands to expedite the development of successful therapeutic applications.

The extensive examination of HLA-B15 ligands, as described herein, enhances understanding the rules that govern natural class I peptide presentation and is secondary evidence of the success and usefulness of the methodology for producing soluble MHC class I and II molecules described and claimed herein. By first building upon the traditional foundations provided by pooled Edman motifs, the data from over 400 individual peptides characterized from B*1501, B*1503, B*1508, B*1510, and B*1512 subsequently indicated that queries for potential epitopes specific to these allotypes would benefit from being optimized in three ways. First, although nonamers represent half of the ligand population, the other 50% of peptide epitopes range down to 7 and up to 12 amino acids in length. Second, effective N-proximal anchor requirements need not be strictly imposed at P2. Third, searches for ligands should weigh C-terminal/proximal sequence matches even more heavily than those of the N-region. The third trend revealed represent, the most substantial of the revised search criteria, since both length variations and lower sensitivity due to the diminishing returns and increasing backgrounds inherent to successive Edman sequencing cycles can C-regional motif trends. As stressed earlier, examples of this bias were evident here in both: (i) the stronger preference by B*1510 for peptides terminating in Val (absent from the pooled motif) rather than Phe (present in the pooled motif); and (ii) the inability of motifs to effectively reflect C-proximal auxiliary anchors.

To illustrate the potential consequences of applying the modified search parameters described, the EBV structural antigen gp85, which has recently been implicated using a murine model as a favorable target against which protective CTLs might be generated, was examined in the context of B*1501 to identify: (i) nonameric epitopes with motif-prescribed P2 and P9 occupancies; (ii) length variant epitopes with motif-prescribed P2 and P9 occupancies; and (iii) nonameric epitopes with flexible P2 occupancy. Since only these three categories of ligands were designated, the inquiry was not exhaustive. However, the information extracted showed that, of the 98 possible epitopes identified, only the 22% within the first column would be placed under further experimental consideration if pooled motifs alone were applied in the search. This is not to imply that the data in this category is invalid but that it might be considerably incomplete for later applications. For example, if either of the AMTSKFLMGTY₁₇₂₋₁₈₂ (varying by length) or the SAPLEKQLF₁₂₃₋₁₃₁ (varying by P2 occupancy) peptides was demonstrated to elicit a more effective antigen-specific CTL response than any of the nonamers bearing standard motif P2/P9 assignments, this knowledge is pivotal to subsequent vaccine design; even if the two designated peptides evoked responses only equivalent to some of the nonamers, their non-motif length and/or sequences discrepancies could prove superior in conferring the ability to overlap multiple allotypes in addition to B*1501. This is advantageous since a vaccine consisting of a single or limited number of peptide specificities could theoretically be effective for protecting populations differing in HLA type.

The majority of information collected from examining divergent HLA-B15 allotypes of sHLA molecules recombinantly produced according to the methodology of the present invention demonstrates that similar and occasionally identical peptide ligands are presented by the different B15 molecules so long as polymorphisms do not alter C-terminal anchoring pockets and while an N-proximal ligand residue can be subsequently anchored within the binding groove. Supporting data furthermore indicates that these principles additionally extend beyond the HLA-B15 allotypes described in specificity herein. Specifically, unpublished results by Ghosh and Wiley (noted in Bouvier and Wiley 1994) indicate that an octamer has been observed to successfully bind a class I molecule by its C terminus despite being shown through x-ray crystallography to not even reach the N-terminal pocket of the binding groove. In addition, a recently-described HIV-gag₁₉₇₋₂₀₅ CTL epitope presented by murine class I K^{d} fails to show a motif-prescribed Tyr at P2 and instead associates stably through its conserved C terminus and an N-proximal preference for Gln at P3 (Mata et al. 1998). The rules established here through examination of hundreds of natural ligands from B*1501, B*1503, B*1508, B*1510, and B*1512 indicate that such occurrences may be more commonplace than exception, as both of these examples appear in agreement with the model.

A step in developing therapies intended to elicit protective CTLs requires the selection of pathogen- and tumor-specific peptide ligands for presentation by MHC class I and class II molecules. Binding/reconstitution assays provide information that is biased due to their technical inconsistency and/or in vitro nature, while Edman sequencing of extracted class I peptide pools generates “motifs” that indicate that the optimal peptides are nonameric ligands bearing conserved P2 and P9 anchors; motifs have frequently been used to provide the search parameters for selecting potentially immunogenic epitopes that might be successfully presented by particular allotypes. Therefore, to test the hypothesis that natural presentation overlaps exist despite the presence of various polymorphisms within the class I binding groove and thus determine how well pooled motifs actually represent their endogenously-derived constituents, ligands were purified from different sHLA molecules produced in hollow-fiber bioreactors, mapped by RP-HPLC and NanoES-MS, and sequenced by NanoES-MS/MS, all according to the present methodology.

Production of sHLA provides an efficient means of extracting large quantities of endogenous peptide ligands for the subsequent analyses, and comparative ion mapping of peptides extracted from distinct class I allotypes is a reliable method for detecting potential ligand overlaps. NanoES-MS/MS analysis then allows for sequence characterization to identify the overlap status of individual ion matches. The strategy developed to address overlap identification is additionally pertinent beyond the uses described herein. For example, similar mapping studies would be performed, with the primary intent instead of characterizing differences between maps, such as between pathogenically infected versus uninfected cell lines; the data obtained could contribute to identifying optimal vaccine epitopes. Successfully locating differences in a similar manner between B*1501 and B*1512 ion maps, as discussed herein, effectively supports this application.

Systematically mapping and characterizing 449 ligands from the related molecules B*1501, B*1503, B*1508, B*1510, and B*1512 demonstrates overall that the peptides bound by these allotypes: (i) vary in length from 7 to 12 residues; and (ii) are more conserved at their C termini that at their N-proximal positions. Flexibility at P2 in particular appears to arise at least in part from the combined effects of distinct steric and charge biases imposed respectively by α-helical and β-sheet structural residues throughout α₁ and α₂ of the various HLA-B15 molecules, while it is postulated that C-terminal preferences are influenced by tapasin-moderated loading selection within the ER.

Although not predictable from the pooled Edman motifs, the comparative peptide mapping strategy succeeded in identifying endogenously processed ligands which bind variously across the allotypes B*1501, B*1503, B*1508, and B*1512, but not B*1510. Overlapping peptide ligands appeared to favorably bind the first four B15 molecules since these allotypes share identical C-terminal anchoring pockets, whereas B*1510 diverges in this region. Endogenous peptide loading into the HLA-B15 allotypes therefore requires that a conserved C terminus be firmly anchored in the appropriate specificity pocket while N-proximal residues act more flexibly in terms of both location and sequence specificity to anchor the ligand into this binding groove region. Subsequently, the choice of allele-specific and/or overlapping peptide epitopes for CTL recognition may thus be contingent upon performing queries strongly based upon conserved C-terminal anchors.

Thus, this methodology proves that ligands derived from sHLA produced according to the methods described above, enables one of ordinary skill in the art to generate immense amounts of data concerning peptide ligands and the motifs of peptide ligands which are bound by HLA molecules. As previously mentioned, such data can generally be broken out into several types of data sets, each having particular advantages and disadvantages. Each type of data set will be discussed in turn.

### sHLA Ligand Data Sets Capable of Being Generated

As mentioned hereinabove, the use of the sHLA production methodology coupled with the sHLA ligand isolation and identification methodology as outlined, results in several categories of sHLA ligand and sHLA ligand motifs which form the core of the sHLA ligand database of the present invention. Each of these categories or data sets of ligand sequences and/or motifs is discussed in detail hereinafter.

### Identification and Sequencing of Individually Identified sHLA Ligands

The amino acid sequencing of an individual peptide requires that the desired peptide be free of contamination by other peptides. The presence of multiple peptides results in two or more peptides being simultaneously sequenced and ambiguity concerning which amino acids comprise the sequence of which peptide in the mixture. It is therefore common to separate the thousands of peptide ligands eluted from a class I molecule. (Huczko, E. L., et al., Characteristics of endogenous peptides eluted from the class I MHC molecule HLA-B7 determined by mass spectrometry and computer modeling. J. Immunol., 1993. 151: p. 2572-2587 which is expressly incorporated herein by reference in its entirety)

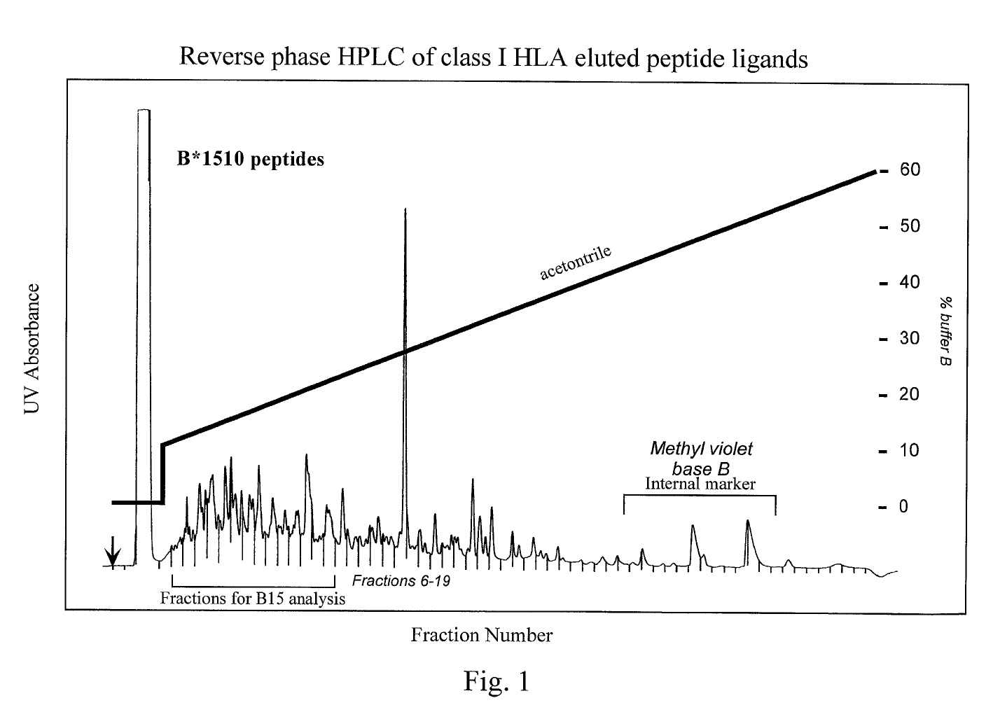

One common means of separating peptides prior to amino acid sequence analysis is the use of reverse phase high pressure liquid chromatography (HPLC). As described in the submotif section of this document, gradual elution of the peptides from the reverse phase HPLC column leaves one to a few peptides coming off the column at any given point in time. For this project, we collect one minute fractions that contain from 100-300 peptides per fraction. The flow rate of the HPLC is 100 microliters per minute such that each fraction contains approximately 100 microliters. The 100 microliters is speed vacuum concentrated to 25 microliters. Four microliters of this is then loaded into a nano spray needle and electrosprayed into a PESciex ESI/TOF mass spectrometer. In general, this 4 microliters can be gradually sprayed into the ESI/TOF mass spectrometer over a period of 30 minutes. A reverse phase HPLC graph is shown for eluted peptide ligands from HLA allele B*1510 in FIG. 1.

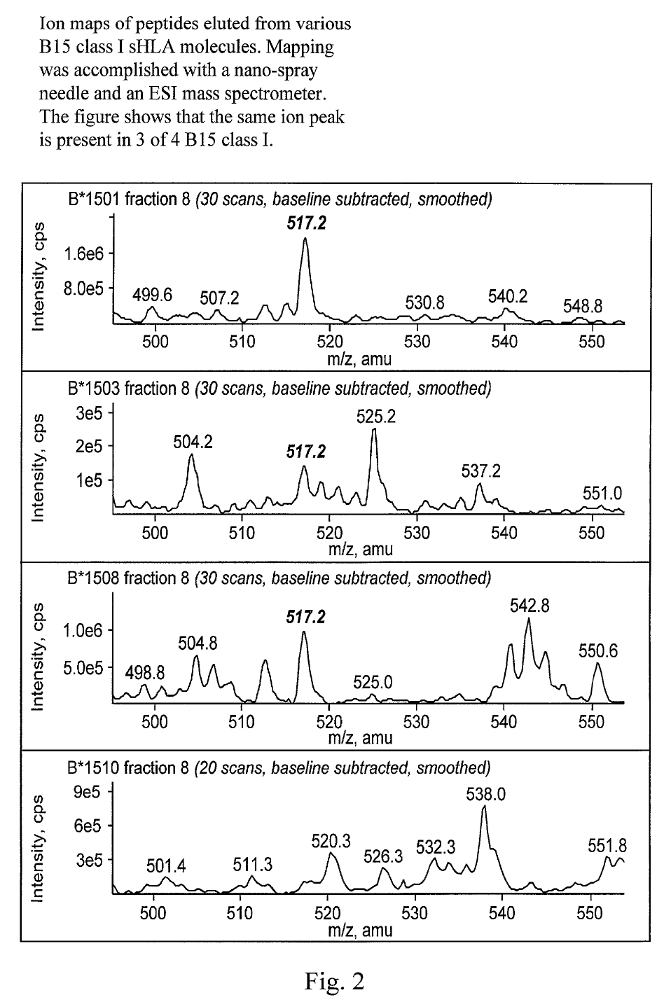

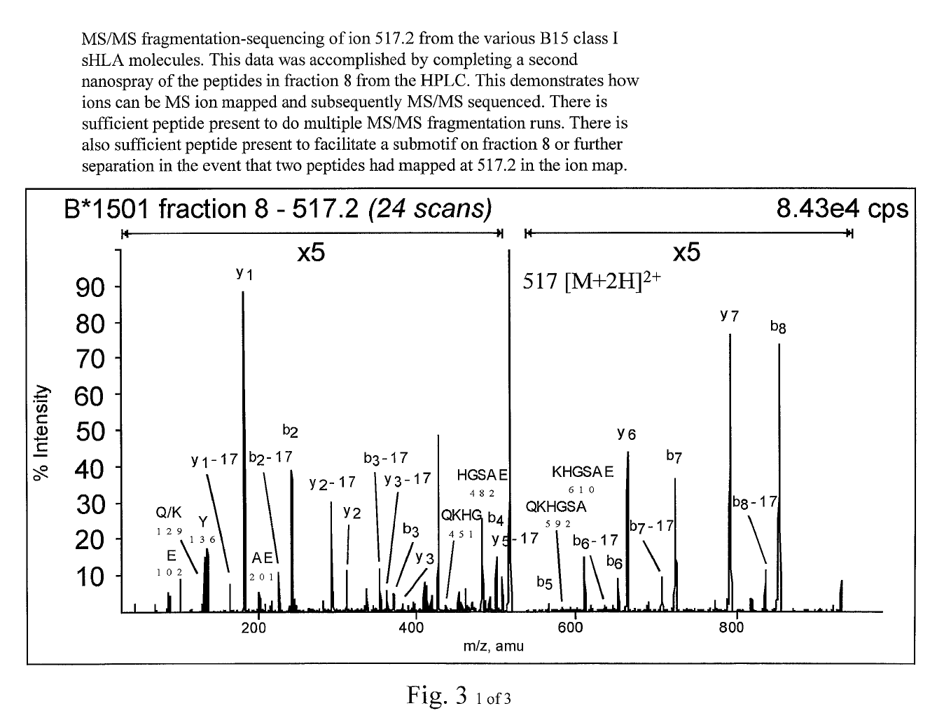

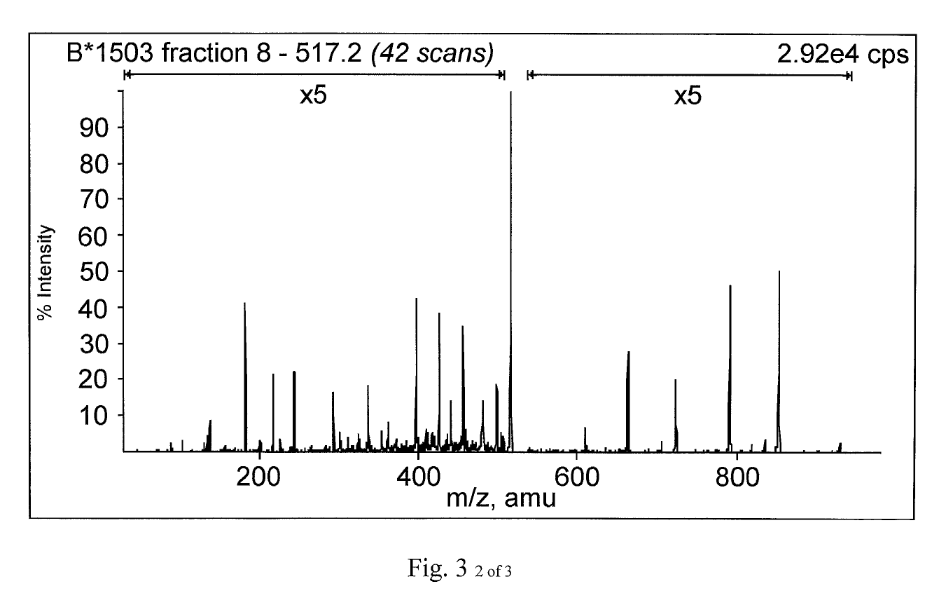

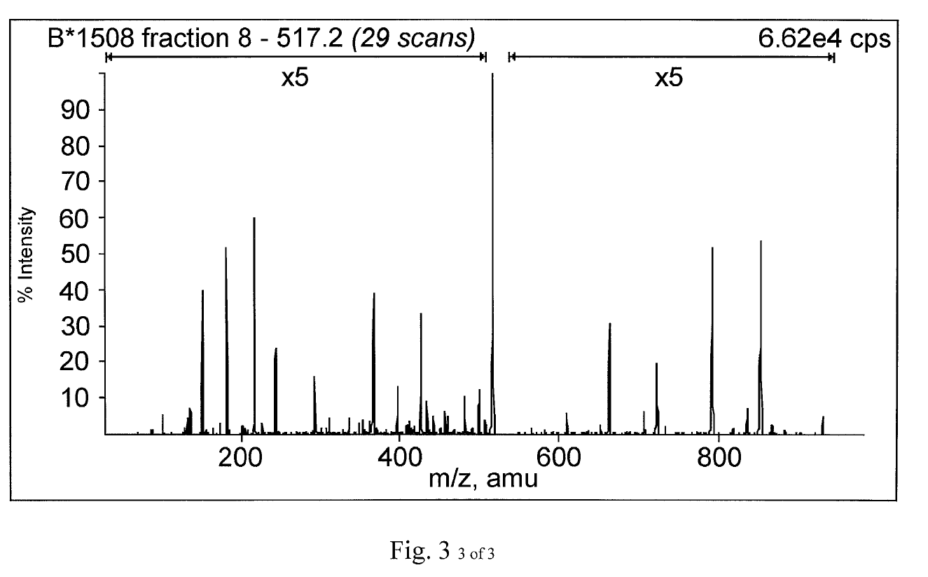

The 200 or so peptides in the HPLC fraction are separated by the ESI/TOF based upon their charge to mass ratio. This mass spectrometric ion mapping therefore adds a second and third dimension of separation based upon charge and mass. The resulting peaks on the ion maps therefore represent a single peptide. A resulting ion map is shown in FIG. 2. These single peptides can then be sequenced by switching the mass spectrometer from ion scanning mode to the MS/MS peptide sequencing mode. A ms/ms fragmentation sequencing of an ion is shown in FIG. 3. The switch from scanning to peptide fragmentation-sequencing mode on the mass spectrometer can be made manually or automatically by putting the machine into it's independent data acquisition mode (IDA). Manual data acquisition results in the fragmentation-sequencing of approximately 10 peptides while IDA can sequence the hundreds of peptides in the mixture in the 30 minute spray.

Note that the HPLC can be integrated with the mass spectrometer and peptides can be ion mapped and sequenced directly as they elute from the HPLC column. This has the same effect of separating peptides prior to MS/MS fragmentation-sequencing. An advantage of direct HPLC-MS ion mapping-MS/MS is that fractions need not be collected. The disadvantage is that little to no sample at this exact time point remains for further analysis; it is difficult to reproduce this exact time point for data reanalysis or for changing parameters. The direct HPLC-MS approach is favored by those with little peptide for analysis while sufficient peptide facilitates reanalysis.

The procedures to produce and fractionate the peptides are outlined by Prilliman, K. R., Lindsey, M., Zuo, Y., Jackson, K., Zhang, Y., and Hildebrand, W. H., Large-Scale Production of Class I Bound Peptides: Assigning a Peptide Signature To HLA-B*1501. Immunogenetics, 1997. 45: p. 379-385; Prilliman, K. R., et al., Complexity among constituents of the HLA-B*1501 peptide motif. Immunogenetics, 1998. 48: p. 89-97; Prilliman, K. R., et al., HLA-B15 peptide ligands are preferentially anchored at their C termini. J Immunol, 1999. 162(12): p. 7277-84; and Hickman, H. D., et al., C-terminal epitope tagging facilitates comparative ligand mapping from MHC class I positive cells. Hum Immunol, 2000. 61(12): p. 1339-1346 (each of which is herein expressly incorporated by reference in its entirety) and in the submotif section of this document. Briefly, sHLA equivalent to 10 mg (and having peptide ligands bound thereto) is produced in hollow fiber bioreactors. This sHLA is affinity purified using the W6/32 antibody specific for the native class I heavy protein. The column is washed and the peptides, class I heavy chain, and the beta-2 microglobulin light chain are eluted from the column in a denatured state with acetic acid. Peptide is separated from beta-2 microglobulin and class I heavy chain by size exclusion. The peptide is concentrated, quantitated, and 200 micrograms of peptide is loaded onto a C-18 reverse phase high pressure liquid chromatography (HPLC) column. The peptides are eluted from the HPLC with an increasing gradient of an organic solvent, in this case acetonitrile. The purpose of the reverse phase elution is to gradually elute peptides from the column such that the approximate 10,000 peptides are eluted over a period of time. In this case the 10,000 peptides are eluted in a period of approximately 40 minutes. This period of time can easily be shortened or lengthened.

The separated peptides can be immediately mapped and MS/MS sequenced using IDA on the mass spectrometer. This is accomplished by directly linking the HPLC to the mass spectrometer. Alternatively, the HPLC fractionated peptides can be gathered into tubes. For the data shown FIGS. 1-3, 1 minute fractions were collected of approximately 50-100 microliters each. Portions of each fraction were subjected to nanospray ESI/TOF ion mapping. Ions of interest can be selected and sequenced during the ion mapping using IDA. Alternatively, the ion maps can be analyzed, ions of interest selected, and a second nanospray ESI/TOF can be accomplished with an additional 4 microliters of sample and the mass spectrometer set in the MS/MS fragmentation-sequencing mode.

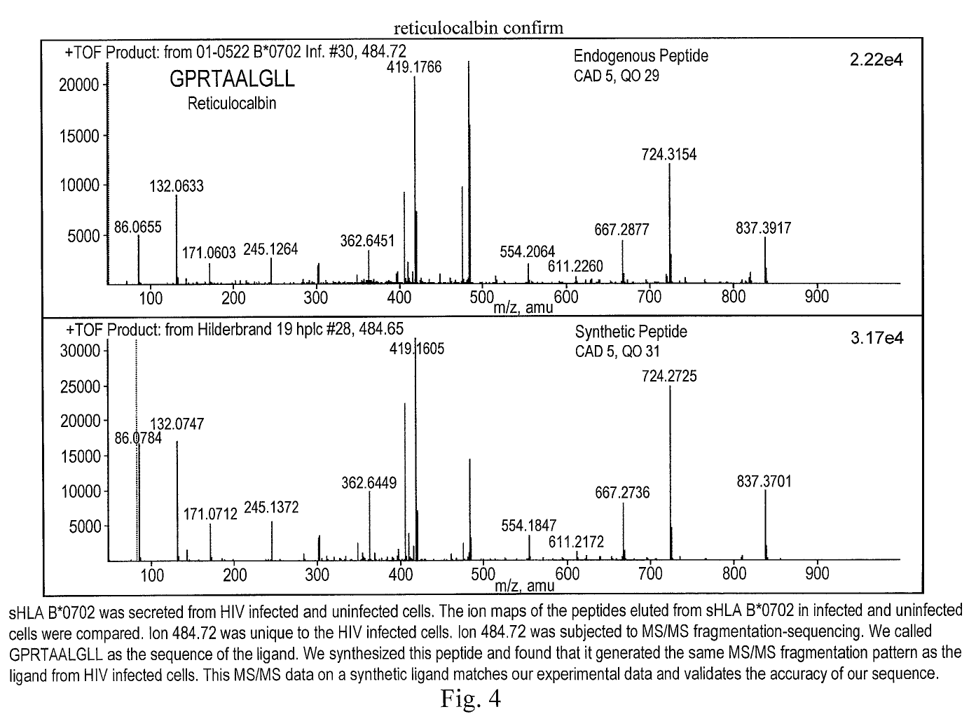

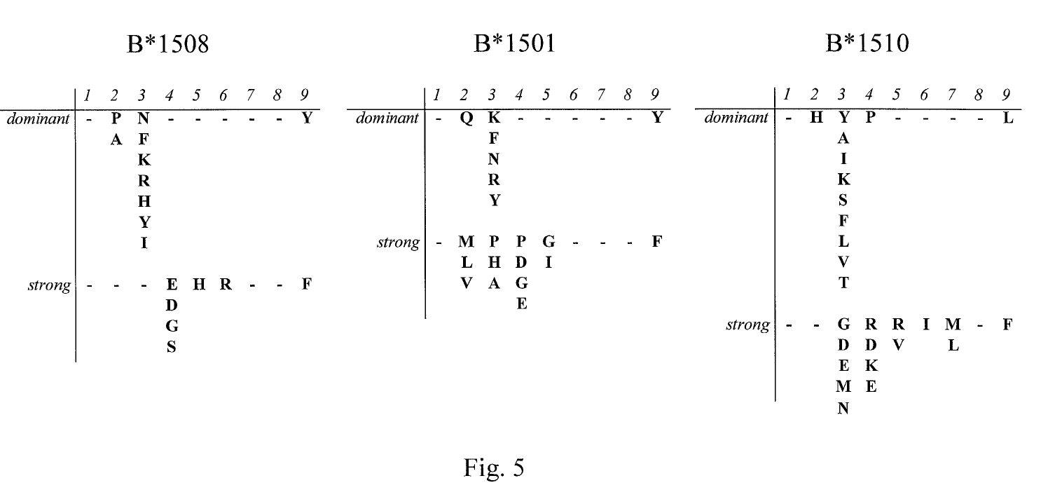

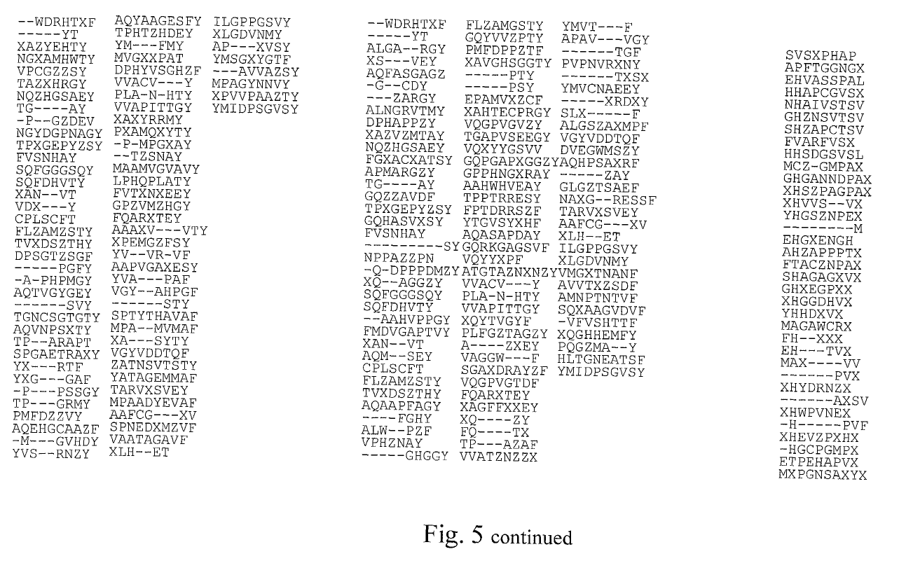

Hundreds of peptides have been sequenced with this approach by the present inventors. The sequence of the interpreted amino acid sequence can then be confirmed by synthesizing a peptide corresponding to the interpreted sequence. This synthetic peptide can then be compared for its HPLC elution profile and its MS/MS fragmentation-sequencing pattern to see if it matches the original. A match validates the MS/MS amino acid sequence assignment. FIG. 4 demonstrates the identification and sequencing of peptide ligands found in an infected cell (HIV) with respect to an uninfected cell. Similarly, FIG. 5 graphically represents deduced peptide ligand motifs for B*1508, B*1501, and B*1510 and the individually identified and sequenced peptide ligands used/discovered which make up the basis of the motifs.

### Motifs of sHLA ligands from Small Pools (John Udell hypothesis)

Another utility of having large amounts of peptide is that extended motifs are obtained. A motif represents up to 10,000 peptides, with many possible amino acids at each position in peptide ligand. In some instances a majority of the peptide will have a predominant amino acid at a position. For example, an R might predominate at P2 in the peptides bound by a given HLA molecule. In the motif, the R will show the strongest signal at P2. However, other subdominant amino acids at P2 of the peptide ligands may be the most important in terms of generating an immune response.

It has been demonstrated that determinants that are subdominant (i.e. of lesser importance for immunity) may represent the predominant peptide ligand in the HLA molecule (Yewdell et al. Immunodominance in Major Histocompatibility complex Class I-Restricted T Lymphocyte Responses, Annual Review of Immunology, Volume 17,1999, pages: 51-88, which is expressly incorporated herein in its entirety). A motif derived from a limited amount of peptide might only show the most prevalent peptide instead of the most important peptide in terms of immune responses. A predictive algorithm using such a limited motif would then select subdominant peptide ligands preferentially over dominant peptide ligands. Population of a database with “short” motifs derived from limited peptide would therefore result in predictive algorithms selecting peptides that are not immunodominant.

An advantage of producing sHLA in milligram quantities is that milligram quantities of peptide ligands are also available. Motifs based upon these peptides will represent peptides that are not the predominant binding peptide. These predominant peptides will appear in motifs using plentiful peptide because the Edman sequencing method upon from which motifs are derived requires that an amino acid signal above background levels be recorded before that amino acid can be entered into the motif. Lesser amounts of peptides leave only the strongest amino acids at each position to be entered in the motif. However, establishing motifs from larger amounts of peptide allows a hierarchy of amino acids at each position in the peptide to be clearly established.

Predictive algorithms must account for the fact that the best binding or most prevalent peptide bound by HLA molecules is not necessarily the most important in terms of mounting an immune response. Using sHLA to establish motifs allows less prevalent peptide ligands to be included in a motif and therefore the database upon which predictive algorithms are founded. Such extended motifs empower predictive algorithms with the ability to identify less prevalent, but potentially immunodominant, peptide ligands for vaccines and other uses.

### Submotifs of sHLA Ligands—Fractionation of Large Pools of sHLA Ligands

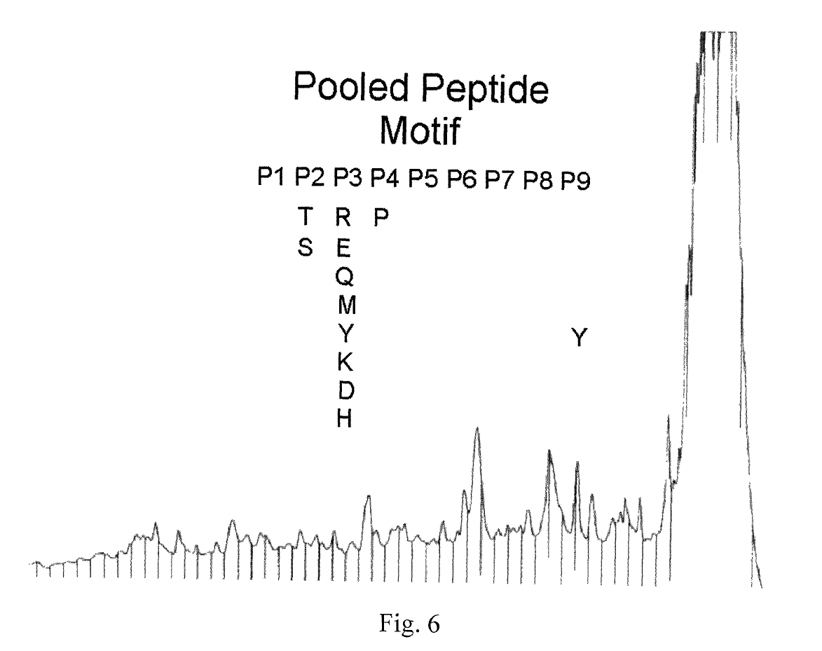

The utility of submotifs lies in their ability to better identify peptide fragments of a protein that will bind to HLA molecules. For example, a typical pooled motif is derived from as many as 10,000 peptides. A T might often be found at P2, an R at P3, and a Y at P9 in the pooled peptides. However, one does not know if the—TR- - -Y sequence is ever found in a linear fashion: i.e. Does a P2 T ever travel with a P3 R and a P9 Y on one peptide. Characterizing thousands of peptides at once makes it difficult and often error prone to string together a linear sequence to search. FIG. 6 graphically demonstrates a pooled peptide motif.

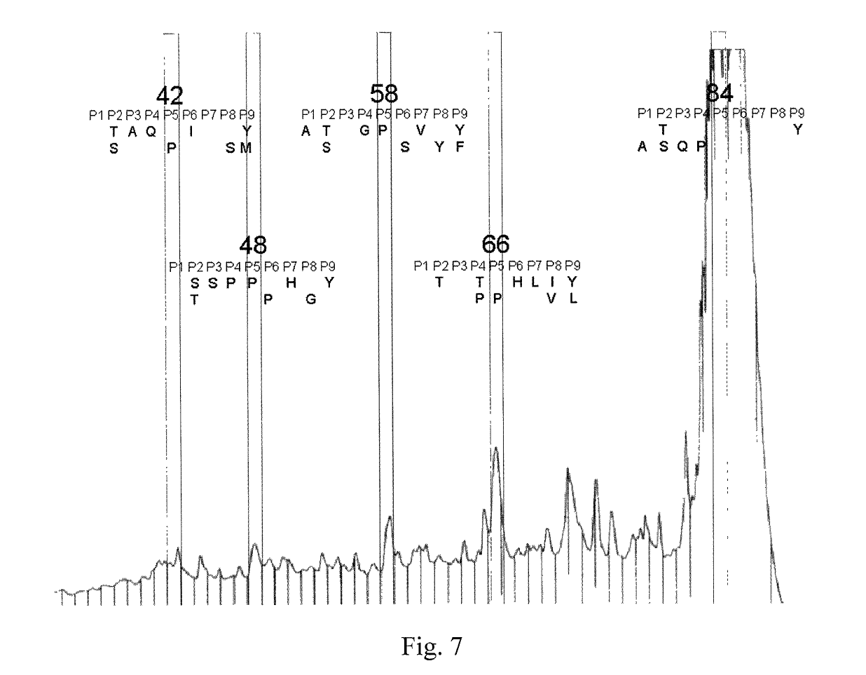

Submotifs result from the sequencing of less than 1000 peptides, usually 200-300 peptides. The resulting data tends to differ from the whole pooled motif, and one is more likely to realize what is associated in a linear fashion. For example, as shown in FIG. 7the P2 T and P9 Y are found in the submotif, but the P3 R is not. Rather, a P3 A is in the submotif, but this P3 A did not show up in the whole pooled motif. The submotif therefore identifies amino acids that can be missed in the whole pooled motif in a way that indicates in a more linear fashion the amino acids that might travel with this P3 A which is missing from the pooled motif. Thus, the submotifs as shown in FIG. 7 are capable of accurately defining linear relationships between amino acids at specific positions within any particular motif.

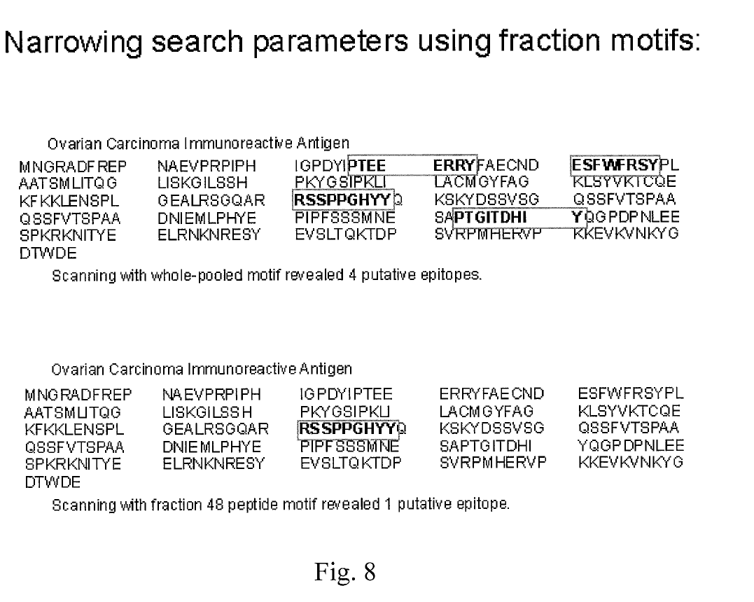

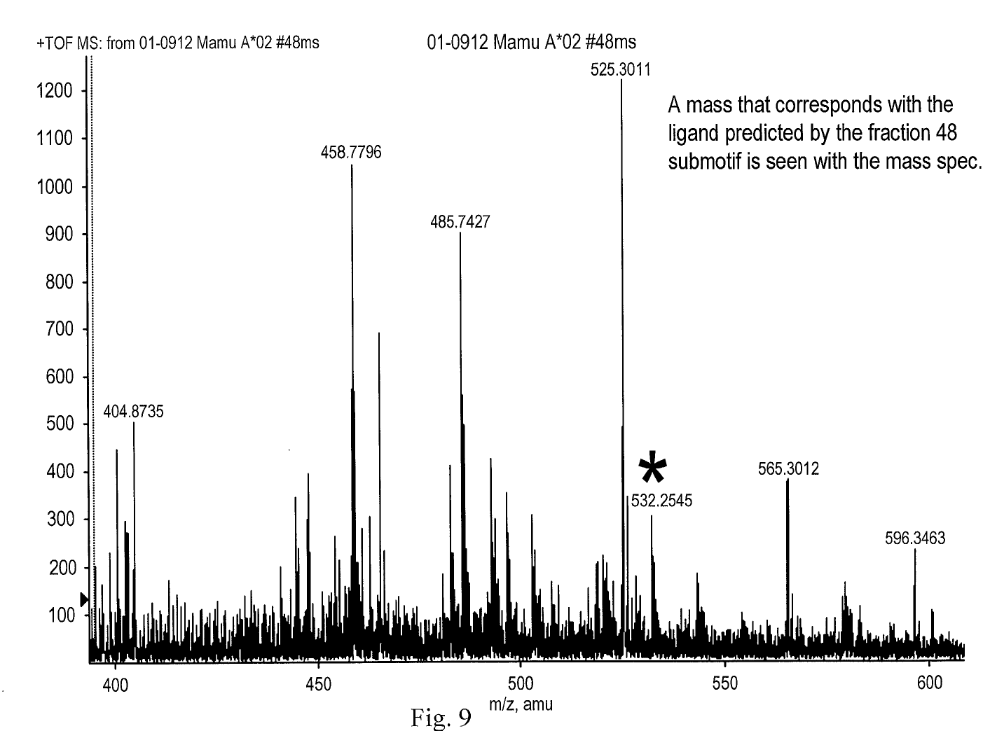

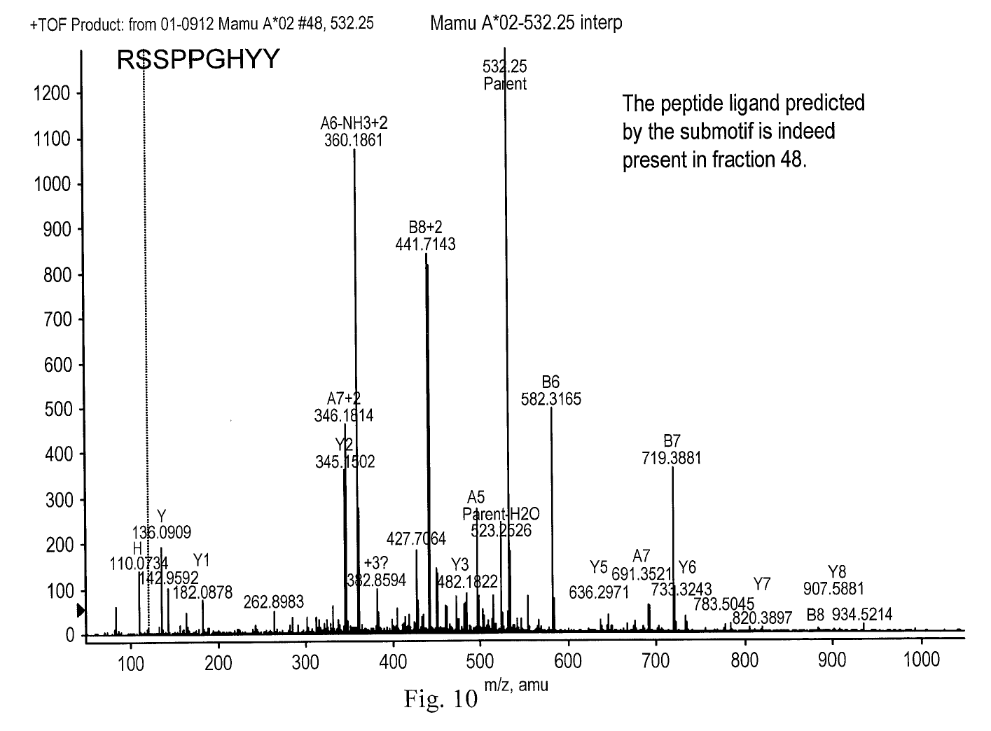

An example of how this submotif can be used to find HLA binding ligands derived from a protein is described hereinbelow. For example, if one uses the whole pooled motif to search Ovarian Carcinoma Immunoreactive Antigen (OCIA), there are several possible matches that may or may not bind (see e.g. FIG. 8). One must then try to find the possible matching peptides in an incredibly complex mixture of peptide ligands, some of which may have the same size as the possible matches. In contrast, if a search is conducted with the fraction 48 submotif of OCIA, one ligand is identified that is a strong match for the submotif. That ligand can then be found in fraction 48. For example, in the OCIA paradigm, the potential ligand was found in fraction 48 FIG. 9. You have therefore reduced the number of possible database hits, and you know where to look for the possible match: in fraction 48. Furthermore, by reducing the complexity to a few hundred peptides in fraction 48, you are much less likely to find multiple peptide ligands at the mass of the putative match. In this case we were able to use the submotif to scan OCIA and find a fragment bound by the class I molecule from which the peptides were eluted (FIG. 8). The MS/MS sequence data illustrates that RSSPPGHYY was found from OCIA in fraction 48 (FIG. 10).

The separated peptides can be immediately mapped and MS/MS sequenced using independent data acquisition on the mass spectrometer. This is accomplished by directly linking the HPLC to the mass spectrometer. Alternatively, the HPLC fractionated peptides can be gathered into tubes. For the data shown here, 1 minute fractions were collected of approximately 50 microliters each. Portions of each fraction are subjected to Edman sequencing, yielding a submotif. The remainder of the fraction is subjected to MS ion mapping after which particular peptides are sequenced from that fraction with MS/MS.

Resulting MS/MS spectra are interpreted with Biomultiview or other software which assists in the interpretation of MS/MS fragmentation patterns. Once a partial amino acid sequence tag or complete peptide sequence has been assembled, software packages such as Mascot or Protein Prospector are used to search available protein databases in order to identify the source of the sequenced peptide. The sequence of the peptide can be confirmed by synthesizing the interpreted peptide and confirming its elution pattern on the HPLC and its fragmentation pattern on the mass spectrometer with MS/MS match that of the interpretated data.

By using submotifs, linear relationships between amino acids in a deduced motifcan be accurately identified. Utilizing such submotifs allows forthe search and testing of potential peptides which may stimulate an immune response.

### Extended Motifs of sHLA Ligands—Unfractionated Large Pools of sHLA Ligands

Utilizing large amounts of peptides eluted from sHLA molecules produced according to the methodology described herein, one of ordinary skill in the art is capable of producing extended motifs.

Pooled Edman motifs of peptide ligands eluted from class I molecules were originally performed as a means to get a feel for the amino acid sequence nature of the peptides bound to a particular class I molecule. Each class I molecule may bind up to 10,000 different peptide ligands. Edman sequencing of thousands of class I molecules at once gives a feel for the general trends of the peptide population. However, it does not tell you about the individual peptides in the population.

Class I protein has been traditionally difficult to isolate. Thus, isolating and characterizing the amino acid sequence of one particular peptide ligand is made difficult by the complexity of the mixture and a small amount of protein. For reasons of complexity and protein concentration, motifs emerged as a means for sequencing class I eluted peptide ligands. The amino acid sequence data that results from pooled Edman sequencing can vary in what it tells you about the peptide population.

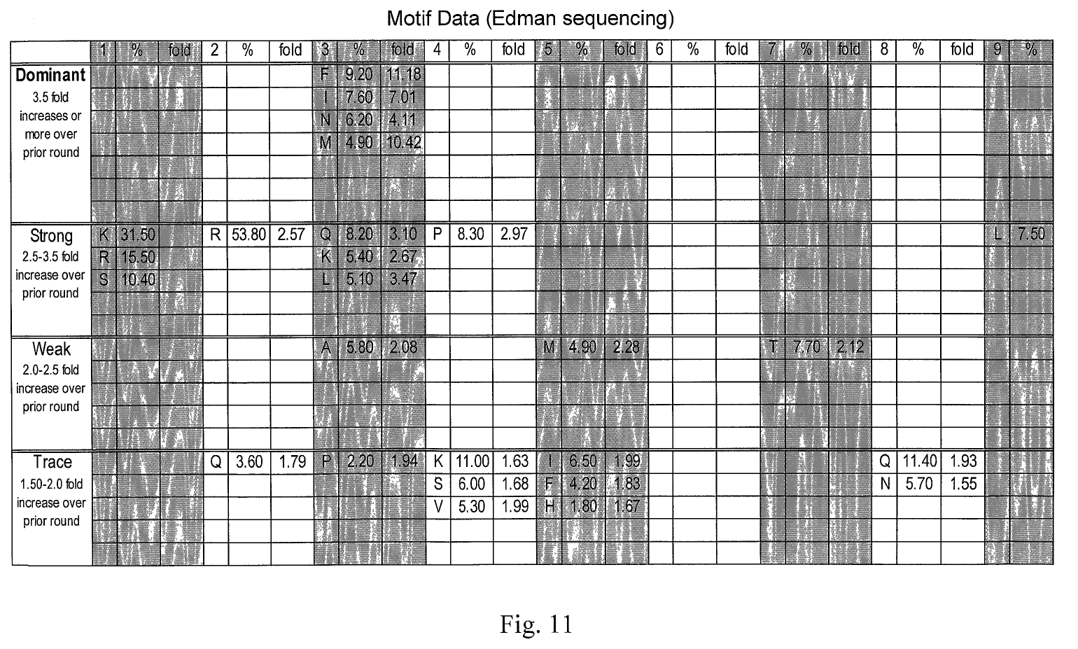

Edman motifs provide more information as to the variability in the peptide population bound by a class I molecule when the motif is established with more than 1000 pico moles of peptide. As peptide concentrations are decreased, the amount of information pertaining to the peptide population also decreases. For example, provided in FIG. 11 is an Edman motif with approximately 1000 picomoles of peptide. Categories in the Edman data of FIG. 11 range from dominant, strong, weak, and trace as noted in the column on the left. As peptide concentrations decrease, the trace, weak, and strong amino acid sequence data will disappear from the table. That is, the signal of these amino acids on the Edman protein sequencer when a higher concentration is used will rise significantly above background levels.

One strength of producing milligrams of individual sHLA includes the fact that antibodies specific for the desired HLA molecule are not needed. This contrasts to detergent lysates which may contain up to six different class I HLA molecules. An antibody specific for the desired HLA molecule may or may not exist. Antibodies specific for particular HLA class I molecules are known to be influenced by the peptide ligands bound by the class I molecule. Therefore, using an antibody to purify a specific class I molecule can bias the peptides characterized according to Solheim, J. C., et al., Binding of peptides lacking consensus anchor residue alters H-2L^{d} serologic recognition. J. Immunol ., 1993. 151(10): p. 5387-5397; and Bluestone, J. A., et al., Peptide-induced conformational changes in class I heavy chains alter major hisotocompatibility complex recognition. Journal of Exp. Med., 1992. 176(6): p. 1757-61, each of which is herein expressly incorporated by reference in their entirety.

Another strength of producing milligram quantities of individual class I molecules is that it facilitates the production of extended motifs. Extended motifs provide additional information concerning the population of peptides which can bind to a class I molecule. One object of the present invention is to provide an HLA ligand dataset that best represents the ligands bound by any class I molecule. Providing more peptide provides more extensive data concerning the ligands that will bind, and this extended knowledge of the class I ligand dataset makes predictive algorithms and linear comparisons more powerful.

A third strength of the extended motifs that result from the Edman sequencing of more than 1000 picomoles of peptide is that the data can be combined with submotifs and individual peptide sequences. Using motifs, submotifs, and individual ligand sequences, the database user and the database designer have a true feeling for the population as a whole, segments of the population that migrate together by hydrophobicity, and the individuals within the population. This data set provides the most predictive power for what will bind to HLA proteins.

### Pathogen Unique Epitopes and Tumor Specific Epitopes

The eluted peptide ligands may also be used to identify, isolate, and sequence peptide ligands which are unique to infected or tumor cells as compared to normal healthy cells. Identifying such unique peptide ligands allows them to be used as vaccines and/or to form the basis of a search for even more powerful and/or selective peptide ligands.

The method of distinguishing infected/tumor cells from uninfected/non-tumor cells is similar to that of producing eluted peptide ligands. The method broadly includes the following steps: (1) providing a cell line containing a construct that encodes an individual soluble class I or class II MHC molecule (wherein the cell line is capable of naturally processing self or nonself proteins into peptide ligands capable of being loaded into the antigen binding grooves of the class I or class II MHC molecules); (2) culturing the cell line under conditions which allow for expression of the individual soluble class I or class II MHC molecule from the construct, with such conditions also allowing for the endogenous loading of a peptide ligand (from the self or non-self processed protein) into the antigen binding groove of each individual soluble class I or class II MHC molecule prior to secretion of the soluble class I or class II MHC molecules having the peptide ligands bound thereto; and (3) separating the peptide ligands from the individual soluble class I or class II MHC molecules.

These methods may, in one embodiment, utilize a method of producing MHC molecules (from genomic DNA or cDNA) that are secreted from mammalian cells in a bioreactor unit. Substantial quantities of individual MHC molecules are obtained by modifying class I or class II MHC molecules so that they are capable of being secreted, isolated, and purified. Secretion of soluble MHC molecules overcomes the disadvantages and defects of the prior art in relation to the quantity and purity of MHC molecules produced. Problems of quantity are overcome because the cells producing the MHC do not need to be detergent lysed or killed in order to obtain the MHC molecule. In this way the cells producing secreted MHC remain alive and therefore continue to produce MHC. Problems of purity are overcome because the only MHC molecule secreted from the cell is the one that has specifically been constructed to be secreted. Thus, transfection of vectors encoding such secreted MHC molecules into cells which may express endogenous, surface bound MHC provides a method of obtaining a highly concentrated form of the transfected MHC molecule as it is secreted from the cells. Greater purity is assured by transfecting the secreted MHC molecule into MHC deficient cell lines.

Production of the MHC molecules in a hollow fiber bioreactor unit allows cells to be cultured at a density substantially greater than conventional liquid phase tissue culture permits. Dense culturing of cells secreting MHC molecules further amplifies the ability to continuously harvest the transfected MHC molecules. Dense bioreactor cultures of MHC secreting cell lines allow for high concentrations of individual MHC proteins to be obtained. Highly concentrated individual MHC proteins provide an advantage in that most downstream protein purification strategies perform better as the concentration of the protein to be purified increases. Thus, the culturing of MHC secreting cells in bioreactors allows for a continuous production of individual MHC proteins in a concentrated form.

The method of producing MHC molecules utilized in the present invention begins by obtaining genomic or complementary DNA which encodes the desired MHC class I or class II molecule. Alleles at the locus which encode the desired MHC molecule are PCR amplified in a locus specific manner. These locus specific PCR products may include the entire coding region of the MHC molecule or a portion thereof. In one embodiment a nested or hemi-nested PCR is applied to produce a truncated form of the class I or class II gene so that it will be secreted rather than anchored to the cell surface. In another embodiment the PCR will directly truncate the MHC molecule.

Locus specific PCR products are cloned into a mammalian expression vector and screened with a variety of methods to identify a clone encoding the desired MHC molecule. The cloned MHC molecules are DNA sequenced to insure fidelity of the PCR. Faithful truncated clones of the desired MHC molecule are then transfected into a mammalian cell line. When such cell line is transfected with a vector encoding a recombinant class I molecule, such cell line may either lack endogenous class I MHC molecule expression or express endogenous class I MHC molecules. One of ordinary skill of the art would note the importance, given the present invention, that cells expressing endogenous class I MHC molecules may spontaneously release MHC into solution upon natural cell death. In cases where this small amount of spontaneously released MHC is a concern, the transfected class I MHC molecule can be “tagged” such that it can be specifically purified away from spontaneously released endogenous class I molecules in cells that express class I molecules. For example, a DNA fragment encoding a HIS tail may be attached to the protein by the PCR reaction or may be encoded by the vector into which the PCR fragment is cloned, and such HIS tail, therefore, further aids in the purification of the class I MHC molecules away from endogenous class I molecules. Tags beside a histidine tail have also been demonstrated to work, and one of ordinary skill in the art of tagging proteins for downstream purification would appreciate and know how to tag a MHC molecule in such a manner so as to increase the ease by which the MHC molecule may be purified.

The method for detecting those peptide epitopes which distinguish the infected/tumor cell from the uninfected/non-tumor cell is a novel approach in the art. The results obtained from such a methodology cannot be predicted or ascertained indirectly; only with a direct epitope discovery method can the epitopes that are unique to infected or tumorous cells be identified. Furthermore, only with this direct approach can it be ascertained that the source protein is degraded into potentially immunogenic peptide epitopes. Finally, this unique approach provides a glimpse of which proteins are uniquely up and down regulated in infected/tumor cells.

The utility of such HLA-presented peptide epitopes which mark the infected/tumor cell are four-fold. First, diagnostics designed to detect a disease state (i.e., infection or cancer) can use epitopes unique to infected/tumor cells to ascertain the presence/absence of a tumor/virus. Second, epitopes unique to infected/tumor cells represent vaccine candidates. Here, we describe epitopes which arise on the surface of cells infected with HIV. Such epitopes could not be predicted without natural virus infection and direct epitope discovery. The epitopes detected are derived from proteins unique to virus infected and tumor cells. These epitopes can be used for virus/tumor vaccine development and virus/tumor diagnostics. Third, the process indicates that particular proteins unique to virus infected cells are found in compartments of the host cell they would otherwise not be found in. Thus, we identify uniquely upregulated or trafficked host proteins for drug targeting to kill infected cells. Finally, the data obtained can be used to push forward drug discovery or vaccine candidates through the use of an sHLA ligand database populated with such data.

Peptide epitopes unique to HIV infected cells are particularly described herein. Peptide epitopes unique to the HLA molecules of HIV infected cells were identified by direct comparison to HLA peptide epitopes from uninfected cells.

As such, and only by example, the present method is shown to be capable of identifying: (1) HLA presented peptide epitopes, derived from intracellular host proteins, that are unique to infected cells but not found on uninfected cells, and (2) that the intracellular source-proteins of the peptides are uniquely expressed/processed in HIV infected cells such that peptide fragments of the proteins can be presented by HLA on infected cells but not on uninfected cells.

The present method also, therefore, describes the unique expression of proteins in infected cells or, alternatively, the unique trafficking and processing of normally expressed host proteins such that peptide fragments thereof are presented by HLA molecules on infected cells. These HLA presented peptide fragments of intracellular proteins represent powerful alternatives for diagnosing virus infected cells and for targeting infected cells for destruction (i.e., vaccine development).

A group of the host source-proteins for HLA presented peptide epitopes unique to HIV infected cells represent source-proteins that are uniquely expressed in cancerous cells. For example, through using the present methodology, it was determined that a peptide fragment of reticulocalbin is uniquely found on HIV infected cells. A literature search indicates that the reticulocalbin gene is uniquely upregulated in cancer cells (breast cancer, liver cancer, colorectal cancer). Thus, the HLA presented peptide fragment of reticulocalbin which distinguishes HIV infected cells from uninfected cells can be inferred to also differentiate tumor cells from healthy non-tumor cells. Thus, HLA presented peptide fragments of host genes and gene products that distinguish the tumor cell and virus infected cell from healthy cells have been directly identified. The present epitope discovery method is also capable of identifying host proteins that are uniquely expressed on or uniquely processed in virus infected or tumor cells. HLA presented peptide fragments of such uniquely expressed or uniquely processed proteins can be used as vaccine epitopes and as diagnostic tools.

The methodology to target and detect virus infected cells may not be to target the virus-derived peptides. Rather, the present methodology indicates that the way to distinguish infected cells from healthy cells is through alterations in host encoded protein expression and processing. This is true for cancer as well as for virus infected cells. The present methodology results in data which indicates without reservation that proteins/peptides distinguish virus/tumor cells from healthy cells.

Class I and class II HLA molecules stimulate protective immune response by binding peptide portions, or epitopes, of a pathogen and presenting these epitopes to immune effector cells. Vaccine architects therefore strive to identify those portions of a pathogen that stimulate protective immune responses; these epitopes must be included in their vaccines. The vaccine architect must know whether the epitopes in their vaccine are bound by HLA molecules and stimulate protective immune responses. Due to the complexity of the HLA complex, the complexity of the peptide epitopes loaded into one HLA molecule, the complexity of the intracellular machinery that loads peptides into HLA molecule, and the in vitro limitations in identifying potential vaccine candidate epitopes, it is often difficult to directly pinpoint and enumerate protective HLA presented vaccine candidates.

Historically, the most effective vaccines have not resulted from a systematic characterization of the pathogen in question. Rather, the pathogen is heat or chemically inactivated, mixed with an adjuvant, and inoculated. By providing the human immune response with as natural a view as possible of the inactivated pathogen, protective immunity is stimulated. In a similar fashion, we propose to use secreted HLA molecules to produce vaccines that accurately reflect the natural spectrum of HLA pathogen derived ligands that occur during infection. A vaccine based upon HLA carrying a natural spectrum of pathogen derived peptides will best mimic the infected state and is therefore best suited to elicit protective T cells.

In addition, upon in vitro systematic identification of antigenic peptides as possible vaccine candidates, individual purified MHC molecules having such antigenic peptides bound therein could be incorporated into a carrier for providing a form of an augmented “natural vaccine” which would mimic the display of the antigenic peptide by an infected cell or a cell which recognizes an exogenous infected environment. Specific epitope loaded sHLA molecules could by placed alone onto a carrier or artificial APC (aAPC) or be placed onto an aAPC along with sHLA naturally loaded with pathogenic peptides. Such a carrier containing the antigenic peptide-MHC complex could be utilized for vaccine development as well as immunomodulation, depending upon the types of other co-stimulatory signal molecules present on the carrier and which are recognized by the immune system to signal alternate pathways of immune responses.

### Soluble HLA Ligand Database

From the production of individual MHC proteins, a database of sequence information of endogenously produced and loaded ligands identified as bound to sHLA has been developed. The premise for such a database is-that providing a larger quantity of peptide epitopes from an individual HLA molecule provides an advantage in terms of the epitope data in the database. For example, characterization of all the ligands (i.e. pooled peptide ligands) from a large quantity of an individual HLA protein provides a better, more extended, peptide motif. In a similar fashion, provision of a sufficient quantity of individual ligands allows the systematic characterization of individual ligands bound by an HLA protein. The advantage of having extended, systematic, peptide epitope characterization is that prediction of vaccine epitopes is based on this data. The better the database, the better the predictive algorithm.

Such a database of endogenously bound and loaded ligands facilitates searching of viral, bacterial, tumor, or human protein sequences for ligands likely to bind a particular HLA class I or class II protein. Such comparative database searches might be run against pooled peptide motifs, or against individual peptide ligands. The search entry might consist of the genomic sequence of a gene/organism, protein sequence of the organism or gene, or particular amino acids, sequences, or peptide sequences of interest. The database algorithm is able to predict the functionality of an unknown protein sequence in terms of endogenous HLA loading and binding.

Out of the core database of individual HLA ligands and extended motifs, an algorithm to identify putative ligands based on motifs and/or ligands in the database is completed. Entries corresponding to the known HLA ligands and extended motifs can consist of, but are not limited to, genomic sequence and protein sequence information. Note that individual peptide ligands entered into the database may represent a portion of a larger protein. The stretches of the larger protein which flank the peptide epitope entered in the database may also be entered in the database and used as part of the search algorithm. Such flanking regions are known to influence production of the peptide ligands. Flanking sequences impact protein digestion into peptide epitopes. Using endogenous peptide epitopes derived from individual sHLA proteins can therefore predict functionality and can easily be developed into a predictive algorithm that is placed either online via the Internet or made available via a private fee for service. Additionally, stand alone programming can be made available to researchers which incorporates the key attributes of the individual MHC protein database.

A searchable sHLA ligand database and epitope prediction software (including linear and predictive algorithms) is also an embodiment of the current technology. Through the use of the soluble HLA, derived from either cDNA or gDNA starting material, pooled and individual endogenously loaded ligands have been obtained and characterized. The methodology for completing this phase is described hereinabove and in the materials found in the co-pending U.S. applications Ser. Nos. 09/974,366 and 10/022,066 which have been explicitly made a part hereof. Motifs of sequence information have also been generated from the sHLA which allows for the categorization of different epitope sequences into broad categories. This information is compiled into a searchable database which allows a user to screen an unknown peptide sequence for potential matches with sHLA ligand (1) discrete sequences or (2) motifs of sequences. Once the database has been searched, matches can be investigated in order to determine the possible functionality of the unknown peptide sequence. Because of the completeness and concentration of the sHLA obtained to date, better sequencing data of numerous endogenously loaded HLA ligands is found in the sHLA ligand database, and by comparison of such ligands to each other and to the genomic sequence, better motifs are also found in the sHLA ligand database.

The systematic, standardized, and normalized characterization of individual peptide ligands aids in the identification of source proteins for the ligands and therefore flanking sequence of the parent protein for the peptide ligands. Such flanking protein sequence from the parent protein is used to predict whether flanking regions located on either side of the putative epitope will enhance formation of the peptide ligand. From this data, an algorithm is developed to identify putative ligands based on extended motifs, individual ligand sequence, and parent protein flanking sequence. Endogenous ligand sequence from sHLA molecules is then incorporated into a predictive algorithm which can search an unknown query (protein sequence or gene sequence) and predict functionality of the unknown sequence. For example, such epitope prediction software is capable of predicting epitopes which will elicit an immune response in humans.

Clinicians and researchers alike have a vested interest in HLA molecules and the peptide ligands they present. In the present invention, i.e. the sHLA ligand database, a novice as well as an advanced user can access HLA ligand and motif information via a graphical interface. This sHLA ligand database is novel in its approach for using server-side Java Technologies such as Java Servlets and JDBC. This sHLA ligand database is also novel in the fact that it is populated with information derived from sHLA. The user can query for ligand and motif information using various parameters such as allele, amino acid pattern, amino acid sequence, T-cell epitope, specific type of protein, etc. The information submitted via the graphical interface is pre-processed by server-side applications to dynamically construct the appropriate query, after which the query is sent to the database. The result is post-processed by the server-side applications, and finally the formatted result is sent back to the user via the graphical interface. As shown in the sample search session provided in parent application U.S. Ser. No.60/270,357, which is expressly incorporated herein by reference, a user of the sHLA ligand database can find reported motif data for the class I MHC molecule A*2402. Additionally, U.S. Ser. No.60/270,357 provides illustrations of how a user of the sHLA ligand database can find peptide ligands which T-cells see in the context of the class I MHC molecule A*0201. Also, U.S. Ser. No.60/270,357 provides a demonstration of how a researcher could determine whether a newly sequenced hepatitis M protein contains a stretch of amino acids that matches with any reported motif or peptide ligand.

Attached to the parent application U.S. Ser. No.60/270,357 and made an explicit part hereof, are printouts of the graphic interface used with the sHLA ligand database. Through use of this interface, a user is able to search individual MHC ligands and motifs. As can be seen, this database is relatively straightforward in design and use. It is the ligand data obtained from sHLA which allows for the complete and comprehensive searching which has been heretofore unavailable. As evidenced by the paper entitled “ASYFPEITHI: database for MHC ligands and peptide motifs,” by Rammensee et al. (1999), the entirety of which is incorporated herein by reference, the creation of the MHC database, once the sequence and motif information is obtained, is straightforward. Examples of such databases can be found at http://bimas.dcrt.nih.gov./molbio/hlabind/and http://134.2.96.221/scripts/MHCserver.dll/home.htm .

### Overview of T Cell Epitope Prediction Algorithms for Use with HIV (Example)

In the past 10 years, several computer-driven algorithms have been devised to take advantage of the alphabetic representation of protein sequence information to search for T cell epitopes. These algorithms search the amino acid sequence of a given protein for characteristics believed to be common to immunogenic peptides, locating regions that are likely to induce cellular immune response in vitro. Given the rapid expansion of sequence data on geographic subtypes (clades) of HIV and individual HIV quasi-species, the application of these algorithms to HIV proteins may significantly reduce the number of regions which would require in vitro testing for immunogenicity, directing research to more promising segments of HIV proteins and thus potentially reducing the time and effort needed to develop HIV vaccines.

Computer-driven algorithms can identify regions of HIV proteins that contain epitopes and are less variable among geographic isolates; alternatively, computer-driven algorithms can rapidly identify regions of each geographic isolate's more variable proteins that should be included in a multi-clade vaccine. Furthermore, computer-driven searches can be weighted to reflect selected HLA alleles that are most representative of geographic populations or subgroups within one geographic area. Computer-driven searches can also be used as a preliminary tool to evaluate the evolution of immune response to an individual's own quasi species.

The first research groups to suggest that computer algorithms based on patterns of amino acids might be used as a tool for discovering T cell epitopes were DeLisi and Berzofsky and Rothbard and Taylor. DeLisi and Berzofsky originally proposed the hypothesis thatT cell antigenic peptides are amphipathic structures bound in the MHC groove, with a hydrophobic side facing the MHC molecule and a hydrophilic side interacting with the T cell receptor. Rothbard and Taylor's algorithm describes a similar periodicity for a smaller number of amino acid residues. The AMPHI algorithm, based on the DeLisi and Berzofsky observations and developed by Margalit et al., has been widely used for the prediction of T cell antigenic sites from sequence information alone.

Algorithms such as AMPHI, which are based on the periodicity of T cell epitopes, have been re-evaluated due to recent crystallographic determination of MHC structures with bound peptides. These peptides were demonstrated to be lying extended in the MHC groove, in non alpha-helical conformations. An explanation of the predictive strength of AMPHI has been provided by Cornette et al., based on the periodicity analysis of a table of motifs compiled by Meister et al. Essentially, AMPHI describes a common structural pattern of MHC binding motifs, since MHC binding motifs appear to exhibit the same periodicity as an alpha helix. More recently, the rapid expansion of information on the nature of peptides that bind to MHC molecules has led to the evolution of a new class of computer-driven algorithms for vaccine development.

### B. Algorithms Based on MHC Binding Motifs

MHC binding motifs are patterns of amino acids that appear to be common to most of the peptides that bind to a specific MHC molecule. For example, a lysine might be required in position N+1 (one amino acid from the amino terminus), and a valine in position N+8, while any amino acid may occur at any of the other positions. In theory, this would explain why MHC molecules are able to present many different peptides from different proteins, yet MHC specificity can still occur. The peptide motif-MHC specificity appears to be due to the interaction of the amino acid side chains of certain conserved “anchor” residues with pockets in the MHC peptide binding cleft.

Identification of T cell epitopes by locating MHC binding motifs in the sequence of a given protein has been shown to be effective when used to identify immunogenic epitopes for malaria and for Listeria monocytogenes, however the number of regions of any given protein that contain single MHC motifs is usually much too large to be of any use for vaccine development. Furthermore, MHC binding motifs appear to be relatively imprecise: only about one-third of peptides containing one of the current motifs that is said to predict binding to a given class I MHC allele have been shown to be bound by that MHC molecule, and in some cases, epitopes that do not contain known MHC binding motifs have been described. This may be due to missing information about the requirements for peptide-MHC interactions, or to errors in the descriptions of MHC binding motifs in the literature. In addition, MHC binding is necessary but not sufficient for a peptide to be antigenic; the peptide-MHC complex must still interact with the TCR of a neighboring cell, allowing the induction of a cellular immune response.

Since 1992, members of the TB/HIV laboratory at Brown University have been working on the development of a computer algorithm that locates MHC binding motifs in amino acid sequences of HIV proteins. In the process of developing this algorithm, it has been demonstrated that MHC binding motifs tend to cluster within proteins. Some of the clustering may be due to the similarity of certain MHC binding motifs to one another, however, dissimilar motifs are also found to cluster. These motif-dense regions appear to correspond with peptides that may have the capacity to bind to a variety of MHC molecules (promiscuous or multi-determinant binders) and to stimulate an immune response in these various MHC contexts as well (promiscuous or multi-determinant epitopes).

The algorithm developed at Brown University (EpiMer) uses a library of MHC binding motifs for multiple class I and class II HLA alleles to predict antigenic sites within a protein that have the potential to induce an immune response in subjects with a variety of genetic backgrounds. EpiMer locates matches to each MHC-binding motif within the primary sequence of a given protein antigen. The relative density of these motif matches is determined along the length of the antigen, resulting in the generation of a motif-density histogram. Finally, the algorithm identifies protein regions in this histogram with a motif match density above an algorithm-defined cutoff density value, and produces a list of subsequences representing these clustered, or motif-rich regions. The regions selected by EpiMer may be more likely to act as multi-determinant binding peptides than randomly chosen peptides from the same antigen, due to their concentration of MHC-binding motif matches.

The MHC binding motif library used by EpiMer for its searches is updated regularly from the literature. This list can be tailored for a number of different types of searches. For example, one can use the entire MHC binding motif library to identify peptides that contain both MHC Class I and Class II binding motifs; one can restrict the list of binding motifs used in the searches to Class I or Class II, and one can tailor the search to the set of MHC alleles of geographic subpopulation or even those of a single individual.

The utility of computer-algorithm driven predictions for in vitro and in vivo research was recently demonstrated in an analysis of peptides predicted by the EpiMer algorithm from Mycobacterium tuberculosis (Mtb) protein sequences. Twenty-seven of 28 EpiMer peptides derived from Mtb proteins stimulated immune responses in peripheral blood cells from Mtb immune subjects. There was a good correlation between the number of motifs per peptide and the number of responders to the peptide in a population of Mtb-infected individuals (p<0.001), and 40 percent of the variation in the relationship between the motifs and the responses could be explained by the presence or absence of MHC binding motifs. As only about a third of peptides that are predicted using single MHC binding motifs are shown to bind and to stimulate immune responses, the relationship between the EpiMer predictions and the number of responders to the peptides was better than expected. It is believed that the selection of regions that are MHC binding motif-dense increases the likelihood that the predicted peptide contains a “valid” motif, and furthermore, that the reiteration of identical motifs may contribute to peptide binding. As can be seen from the Epimer example, the use of predictive algorithms is of immense utility to vaccine developers. Once such predictive algorithms are coupled with sHLA ligand data in the database of the present invention, a robust and reliable source of information and research tools are presented.

Additional MHC binding motif-based algorithms have been described by Parker et al. and Altuvia et al. Parker, K. C., et al., Peptide binding to MHC class I molecules: implications for antigenic peptide prediction. Immunol Res, 1995. 14(1): p. 342-57; Altuvia, Y., O. Schueler, and H. Margalit, Ranking potential binding peptides to MHC molecules by a computational threading approach. J Mol Biol, 1995. 249(2): p. 244-50; Altuvia, Y., et al., A structure-based algorithm to predict potential binding peptides to MHC molecules with hydrophobic binding pockets. Hum Immunol, 1997. 58(1): p. 1-11; Schueler-Furman, O., et al., Structure-based prediction of binding peptides to MHC class I molecules: application to a broad range of MHC alleles. Protein Sci, 2000. 9(9): p. 1838-46; and Schueler-Furman, O., Y. Altuvia, and H. Margalit, Examination of possible structural constraints of MHC-binding peptides by assessment of their native structure within their source proteins. Proteins, 2001. 45(1): p. 47-54, each of which is expressly incorporated herein by reference in their entirety. In these algorithms, binding to a given MHC molecule is predicted by a linear function of the residues at each position, based on empirically defined parameters, and in the case of Altuvia et al., known crystallographic structures are also taken into consideration. DeLisi et al. Neural network method for predicting peptides that bind major histocompatibility complex molecules. Methods Mol Biol, 2001. 156: p. 201-9, which is expressly incorporated in its entirety herein by reference, have proposed an alternative method of determining MHC binding peptides, based on the free energy relationships of each amino acid in the predicted peptide, and analyzing whether the tertiary structure of the peptide conforms to a predetermined MHC binding peptide configuration. Finally, Brusic and colleagues are using artificial neural networks to determine the “rules” for binding to MHC molecules from the array of binding peptides that have been described for each of the human HLA alleles. None of these algorithms have been tested in vivo. Should any of these variations on “motif matching” prove to be accurate predictors of peptides that bind to individual MHC alleles, they may be easily incorporated as subprograms into a clustering algorithm such as that contemplated as part of the present invention, and might improve the algorithm's overall predictive capacity.

Most of the novel computer-driven algorithms depend on published information on MHC binding motifs. One methodological concern when designing a multiple binding motif-based predictive algorithm is the accuracy of the MHC binding motifs used to predict putative epitopes, and thus the overall validity of the motif database. Previously reported motifs are often redefined in the literature, after peptide truncation and alanine substitution experiments are performed; likewise, new emphasis has been placed on the role of protein processing and on the identification of specific amino acid residues at non anchor sites, which interfere with the relative capacities of peptides to bind to the MHC cleft. In addition, several MHC binding motif databases have been constructed. Rammensee et al. in SYFPEITHI: database for MHC ligands and peptide motifs. Immunogenetics, 1999. 50(3-4): p. 213-9, which is incorporated herein in its entirety by reference, have published a motif database, aided by the alignment of actual MHC binding peptides and known T cell epitopes. A new prediction algorithm based on the Rammensee motifs is has been developed in the TB/HIV Research Laboratory. As shown in Brusic, V., et al., Prediction of MHC class II-binding peptides using an evolutionary algorithm and artificial neural network. Bioinformatics, 1998.14(2): p. 121-30; Brusic, V., G. Rudy, and L. C. Harrison, MHCPEP, a database of MHC-binding peptides: update 1997. Nucleic Acids Res, 1998. 26(1): p. 368-71; and Honeyman, M. C., et al., Neural network-based prediction of candidate T-cell epitopes. Nat Biotechnol, 1998. 16(10): p. 966-9, each of which are expressly incorporated herein in their entirety by reference, have taken this MHC motif library concept further by providing an Internet-accessible database of binding motifs and peptides known to bind with affinity to MHC molecules. These databases, however, shared the disadvantages that the information is primarily collected from the public domain, is not representative of the tens of thousands of peptides bound by each HLA allele, and is not standardized nor normalized with respect to data acquisition.

That is, the peptides present therein have typically been obtained by antibody purification of the MHC peptide complex and as the peptides bound to the MHC binding groover affect antibody binding and therefore purification of the complexes, the peptides present in these prior art databases are a biased set of peptides that have been identified because specific antibodies recognize specific MHC-peptide complexes. Therefore, these databases are not representative of the entire population of peptides to which an individual MHC molecule binds.

The purpose of the soluble HLA Ligand/Motif Database of the present invention is to provide the scientific community with access to HLA bound peptide ligands. Knowledge of such ligands can be used to select a peptide fragment of a tumor or viral antigen for use in a T cell eliciting vaccine. In a similar fashion, knowledge of those ligands which bind HLA can be used to design HLA molecules loaded with a particular peptide ligand that will, in turn, suppress or stop an immune response. T lymphocytes react to the peptide presented by HLA molecules, and knowledge of the peptides can be used to modulate T cell responses.

At the present time, there is much variability among the peptide ligands reported for different HLA molecules and, in some cases, among the peptides for a given HLA molecule. This variability among the data found in the literature and current databases results from experimental variation between laboratories and/or between experimental methods. Each lab typically utilizes one antibody to purify A*0201 and a different laboratory will utilize a different antibody to purify A*0201. It is known that antibodies influence the repertoire of peptides observed in an HLA molecule. In addition, many HLA molecules have no antibody specific for them.

Another variation in the peptides is that one laboratory might obtain peptide ligands from B lymphocytes and another lab might transfect and obtain HLA and peptide ligands from a CHO cell. Different cell lines may express different gene products and therefore load different peptides. Using different cell lines also may lead the HLA molecules to compete for available peptide, and with various HLA molecules the competition will differ from cell line to cell line. Thus, the source and purification of the HLA can differ from lab to lab.

A third variable is the empiric methods used to purify the peptides, separate the peptides, and analyze the peptides. One lab might use siliconized tubes to prevent the peptides from sticking to test tubes and another lab might not. These two labs will get different products at the end. Buffers, pipette tips, HPLC columns, and mass spectrometers will also modify the data. For example, MALDI TOF mass spectrometers tend to be able to analyze a different subset of peptides than ESI mass spectrometers.

Use of sHLA molecules produced according to the methods disclosed herein facilitates a standard production method, a standard purification method, and a standardized analysis method. Although various methods of analysis and purification could be developed, the production of sHLA facilitates such standardized methodologies. Standardized methods in turn ensure that the peptides sequenced can be compared between HLA molecules as well as within one HLA specific molecule.

The importance of standardized data is due to the wide variability of HLA molecules in the population. Most individuals have a different HLA type. In order to find a vaccine that works across many different HLA types (i.e. a vaccine that works in many people) the algorithms desire to predict whether a particular vaccine candidate might bind several HLA molecules. The resulting data can be judged as comparable (i.e. the peptide will bind A*0201 but not A*2402) if the dataset is uniform. However, if the A*0201 and A*2402 ligands in the database are not comparable, the utility of the database is diminished because conclusions across various HLA types cannot be made.

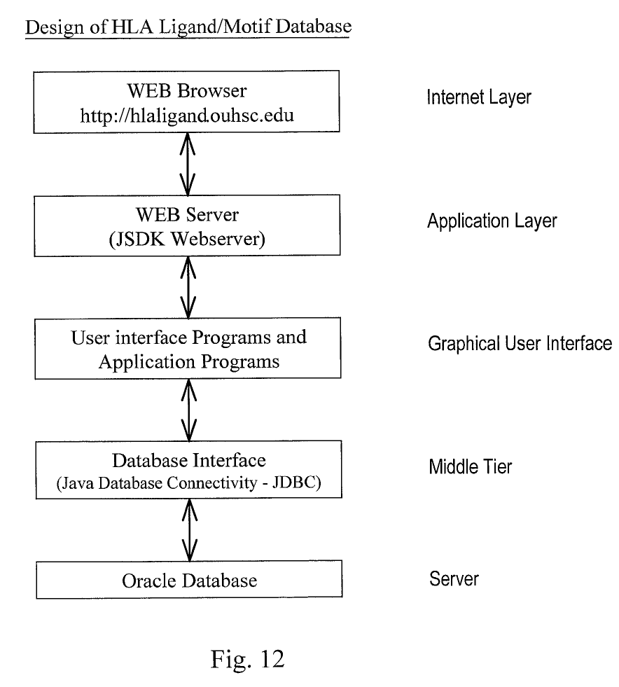

The soluble HLA ligand database of the present invention is shown in schematic format in FIG. 12. A prototype of this database is currently (as of the filing date of this application) accessible and searchable online through http://hlaligand.ouhsc.edu. FIG. 12 shows the design of the entirety of the database. The architecture of this database consists of five layers. First, HLA class I and II peptides are present in an Oracle 8i database which is at the bottom most layer.

An Internet browser that is used to access the database is the first layer, the Internet layer, in the architecture. The website is built using HTML and the input from the webpage is first validated using Java script. After validation the data is given to the lower layers. A web server running on a Windows NT system forms the Application layer for the whole design. Jsdk 2.1 web server runs on the Application layer. The Web server is used to host the website on the Internet.

The graphical User Interface consists of programs written in HTML and Java Servlets. When a peptide sequence is entered for sequence matching or a search is done for a particular allele, the input is given to the Java servlet program which runs an algorithm for each type of search available on the online database. There are five different searches currently available, but as previously discussed, alternate predictive algorithm searching can also be used with the database of the present invention. In the first search, Quick search on ligands and motifs, peptides are displayed for an allele. The next search is Advanced Ligand search, which has options for searching a ligand sequence by source, source type, epitope and by their motif. Advanced pattern search is the next tool in the search engine, which searches the database for ligands and motifs given the amino acid at respective positions. Sequence matching is the fourth tool that matches an entered sequence for peptides in the database. The final search tool is searching by Authors for HLA peptides.

Middle tier in the architecture is written in Java Database Connectivity (JDBC), which is the database interface. JDBC forms an interface between the user interface and the oracle database. It gets the input from the user interface program, connects to the database, converts the inputs into Oracle understandable language, Structured Query Language (SQL), and queries the database for results. The results from the database are then given back to the user interface programs by the JDBC.

The Oracle Database is at the bottom of the architecture, which contains the peptide sequence and other details about each peptide in the form of a table. Oracle 8i is run on a Silicon graphics database server.

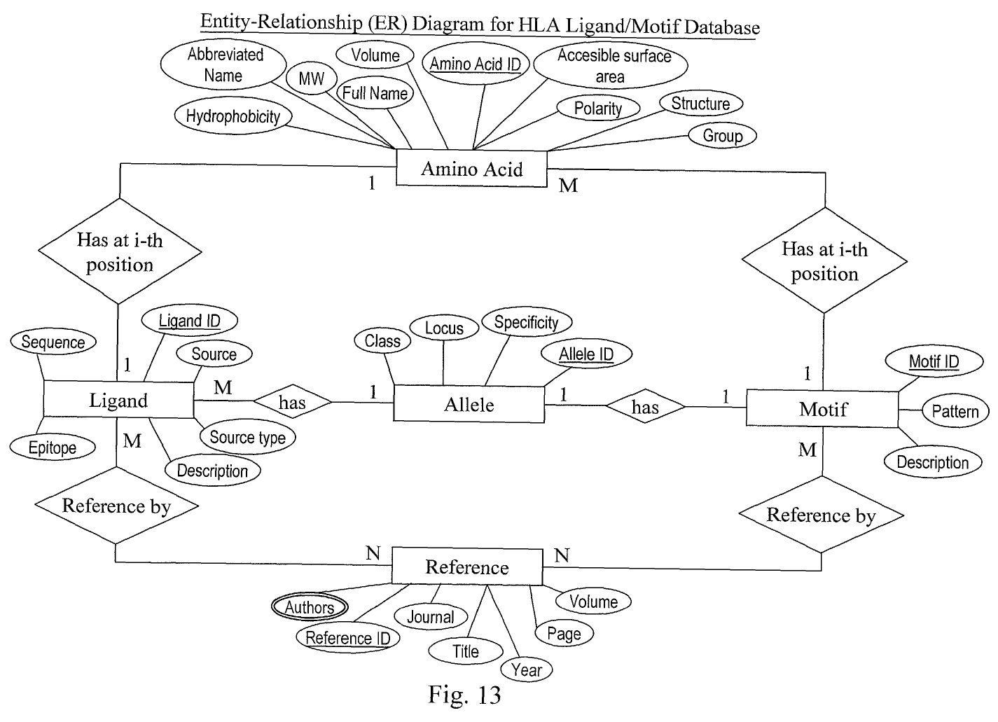

FIG. 13 is an Entity-Relationship (ER) diagram showing the logical view of the database. Relationships between the tables in the database can be found from the ER diagram. HLA class I and II peptides along with their references from the scientific literature are stored in the database as tables. The ER diagram is the first step in designing a good database. Entities are shown using the rectangle symbol in the figure and relationships between entities are shown using the diamond symbol. Entities are the main tables in the database and relationship between each entity is shown using the relationship symbol. Relationship name is written inside the diamond symbol. Oval symbols show the attributes of an entity. Attributes are nothing but a column in a table that is present in a database.

There are five entities in the database namely, HLA class I or class II Allele, Ligands from a particular class I or class II allele, Motifs for a class I or class II allele, References of the scientific journal from where the entry comes, and Amino acids. The Allele entity is related to the Ligand and Motif entities. Ligand and motif entities are related to the amino acid entity and also to reference entity. Allele entity consists of attributes namely Allele name, class, locus and specificity. Ligand entity consists of sequence, source of the ligands (endogenous, T cell epitope, NK epitope, etc.), source type (from a virus, bacteria, etc.), epitope and description of the ligand sequence. Description consists of comments if any present in the manuscript on that particular ligand. Motif entity consists of motif pattern and description. The reference entity consists of Journal name, title of the manuscript in which the peptide is found, volume number, starting page, ending page, year of publication and the authors of the manuscript.

The ER diagram is converted into tables using standard conversion technique used in database management systems. The values in a table can be queried using any query language understandable by Oracle. We use Structured Query Language (SQL), a standard query language.

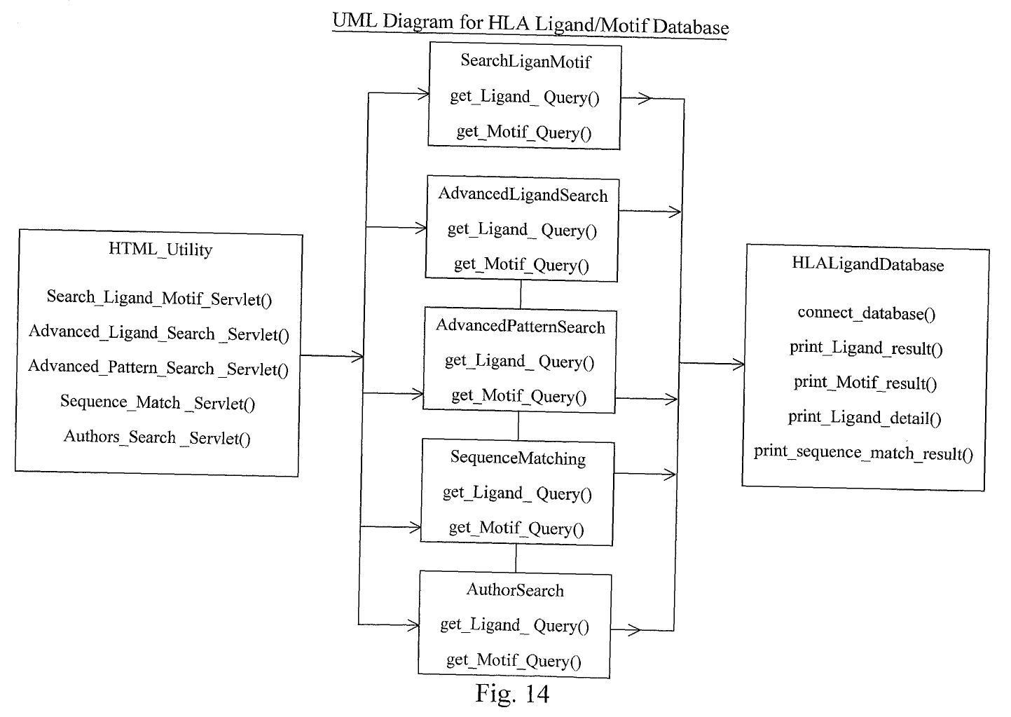

FIG. 14 is an Unified Modeling Language (UML) diagram which helps to specify, visualize, and document models of software systems, including their structure and design. UML suits mostly to object-oriented programming environment and since the present invention is based upon Java, we choose to design using UML. The UML diagram in FIG. 14 shows all the programs and the related functions in a pictorial way.

HTML_Utility is the main program that gets the input from the user and gives to other subprograms for processing queries and displaying the results. It has five important functions namely Search_ligand_motif_servlet, Advanced_ligand_motif_servlet, Advanced_pattern_servlet, Sequence_match_servlet and Authors_search_servlet. Search_ligand_motif_servlet function transfers the control to SearchLigandMotif program, which executes an algorithm to search for ligands and motifs in the database. Advanced_ligand_motif_servlet function transfers the control to AdvancedLigandSearch program that takes the input, does some processing and gives it to the JDBC code that runs inside the program to the database. The getLigandquery and getMotifquery implements a JDBC code to connect to the database and retrieve the results from it. Similarly the AdvancedPatternSearch, SequenceMatch and AuthorsSearch programs implements separate algorithms for searching according to the input and the searches they are supposed to do.

The control transfers from the above sub program to the final main program, which is HLALigandDatabase. The main function of this program is to format the output from the Oracle database to viewable format. Sub-routines present in this program helps in doing its function. All the programs are written in Java servlets. The advantages available in Java environment like operating system independable, security from hacking the database and portability of the code, made us choose the Java environment.

### Linear and Predictive Algorithmic Searching

The sequence of a known peptide ligand itself is not informative; it must be analyzed by comparative methods against existing databases such as the sHLA ligand database of the present invention to develop hypothesis concerning relatives and function. For example: An abundant message in a cancer cell line may bear similarity to protein phosphatase genes. This relationship would prompt experimental scientists to investigate the role of phosphorylation and dephosphorylation in the regulation of cellular transformation.

The General approach of linear searching involves the use of a set of algorithms such as the BLAST programs to compare a query sequence to all the sequences in a specified database. Comparisons are made in a pairwise fashion. Each comparison is given a score reflecting the degree of similarity between the query and the sequence being compared. The higher the score, the greater the degree of similarity. The similarity is measured and shown by aligning two sequences. Alignments can be global or local (algorithm specific). A global alignment is an optimal alignment that includes all characters from each sequence, whereas a local alignment is an optimal alignment that includes only the most similar local region or regions. Discriminating between real and artifactual matches is done using an estimate of probability that the match might occur by chance. Of course, similarity, by itself, cannot be considered a sufficient indicator of function.

The BLAST programs (Basic Local Alignment Search Tools) are a set of sequence comparison algorithms introduced in 1990 that are used to search sequence databases for optimal local alignments to a query. The BLAST programs improved the overall speed of searches while retaining good sensitivity (important as databases continue to grow) by breaking the query and database sequences into fragments (“words”), and initially seeking matches between fragments. The initial search is done for a word of length “W” that scores at least “T” when compared to the query using a given substitution matrix. Word hits are then extended in either direction in an attempt to generate an alignment with a score exceeding the threshold of “S”. The “T” parameter dictates the speed and sensitivity of the search.

The quality of each pair-wise alignment is represented as a score and the scores are ranked. Scoring matrices are used to calculate the score of the alignment base by base (DNA) or amino acid by amino acid (protein). A unitary matrix is used for DNA pairs because each position can be given a score of +1 if it matches and a score of zero if it does not. Substitution matrices are used for amino acid alignments. These are matrices in which each possible residue substitution is given a score reflecting the probability that it is related to the corresponding residue in the query. The alignment score will be the sum of the scores for each position. Various scoring systems (e.g. PAM, BLOSUM and PSSM) for quantifying the relationships between residues have been used.

Positions at which a letter is paired with a null are called gaps. Gap scores are negative. Since a single mutational event may cause the insertion or deletion of more than one residue, the presence of a gap is frequently ascribed more significance than the length of the gap. Hence the gap is penalized heavily, whereas a lesser penalty is assigned to each subsequent residue in the gap. There is no widely accepted theory for selecting gap costs. It is rarely necessary to change gap values from the default.

The significance of each alignment is computed as a P value or an E value. Each alignment must be viewed by a critical human eye before being accepted as meaningful. For example high scoring pairs whose similarity is based on repeated amino acid stretches (e.g. poly glutamine) are unlikely to reflect meaningful similarity between the query and the match. Filters, (e.g. SEG) that mask low complexity regions, can be applied to partially alleviate this problem.

Predictive algorithms, such as Parker's, sypeithi, and the Brown University HIV algorithm may also be built into the present invention as previously discussed. The use of such predictive algorithms is to identify peptide ligands that will bind various HLA molecules. One use of such algorithms is to identify those pieces of a protein that might be bound by, presented by, and immunogenic in a particular HLA molecule. For example, a researcher finds that expression of the Hepatitis X protein corresponds with protective T cell immunity. In order to build a vaccine against the Hepatitis X protein, vaccine researchers may wish to determine which portion of the Hepatitis X protein is presented by HLA and should therefore be in the vaccine.

The Hepatitis X protein may be big while many vaccination strategies aim to use the small peptide fragments. The most expensive and least efficient means of determining which fragment of Hepatitis X to use in a vaccine is to embark upon empiric experiments with all possible Hepatitis X peptides. An alternative to this time consuming and expensive means of empirically identifying immunogenic peptide fragments that bind to HLA is to narrow down the peptides to be empirically tested to those most likely to work. An HLA peptide ligand database provides a means of identifying peptides likely to bind HLA and therefore be immunogenic.

The HLA peptide ligand database can help narrow the choice of vaccine candidates in this example by identifying those portions of Hepatitis X which can bind HLA. Peptide fragments which will not bind Hepatitis X need not be synthesized or tested. This saves time and money and increases the likelihood of success in vaccine development. In this example, the peptide ligand database consists of peptides which are known to bind HLA molecules by the cells natural endogenous peptide loading apparatus.

A predictive algorithm that enriches for peptide ligands that are likely to bind HLA is a logical starting point in vaccine design. Such an algorithm begins the process of sifting through the enormous number of possible peptides that might be used in a vaccine. Such an algorithm utilizes and applies all that is already known of peptide ligands that bind HLA. Utilization of this knowledge in a predictive algorithm allows vaccine developers to build on what is already known rather than repeating it.

The utility of such predictive algorithms is complicated by two factors. The first is the tremendous complexity and variability of the HLA molecules. There are hundreds of different class I molecules which have different peptide binding characteristics. Some of these HLA have been characterized for their peptide ligands and others have not. Therefore, the systematic population of a ligand database requires knowledge of the HLA field and the ability to characterize peptide ligands from a range of HLA molecules.

A second factor which complicates the application of HLA ligand databases is that the peptides from the different HLA molecules have been produced, purified, and characterized with different methods. This means that the peptides for A*0201 may be equivalent to an apple while the peptides for A*2402 in a different lab may be equivalent to an orange. The problem comes when a vaccine developer wants to know if their vaccine will work in both A*2402 and A*0201. A predictive algorithm may indicate the vaccine will work in one molecule and not the other because these two molecules will truly bind the vaccine peptide differently. Alternatively, the vaccine peptide may actually work in both, but because two different laboratories and methods produced the data in the database, the results differ. Thus, the predictive algorithm cannot compensate for peptide ligand data in the database that has been gathered differently and is not equivalent.

Production of sHLA molecules provides a solution for the uniform, systematic, population of an HLA ligand database. Various sHLA molecules can be produced in the same cell line, purified in the same manner, and peptides sequenced the same way. Moreover, production of plentiful HLA protein allows for the systematic characterization of peptides. Extended motifs, submotifs, and the sequencing of numerous individual peptide ligands can be accomplished. Thus, sHLA facilitates the uniform, systematic population of the database as no other system can. Population of a database with these ligands in turn empowers the predictive algorithms to be accurate and consistent.

The example above describes the use of predictive algorithms for vaccine development. Other areas of use for such algorithms include transplantation and autoimmune studies. All areas in which HLA and their peptide ligands trigger immune responses will benefit from a systematically populated HLA ligand database.

Thus, in accordance with the present invention, there has been provided a methodology for producing and manipulating Class I and Class II MHC molecules from gDNA/cDNA and a method for producing soluble Class I or Class II MHC ligands which can be isolated, identified, and sequences and this information can in turn be utilized for vaccine development or immunomodulation. The primary pathway for using such information (as pursuant to the instant application) is a soluble HLA (sHLA) ligand database populated with sequences and motifs generated as above coupled with prediction software/algorithms that fully satisfies the objectives and advantages set forth herein. Although the invention has been described in conjunction with the specific drawings, experimentation, results and language set forth herein above, it is evident that many alternatives, modifications, and variations will be apparent to those skilled in the art. Accordingly, it is intended to embrace all such alternatives, modifications and variations that fall within the spirit and broad scope of the invention.

What I claim is:

1. A method of accessing soluble HLA ligand data stored in a database comprising the steps of:
providing a database containing soluble HLA ligand data stored therein;
providing a means for accessing the database via a remote connection; and
providing a means for searching the soluble HLA ligand data stored in the database via the remote connection.

2. A computer system for a soluble HLA ligand database, comprising:
a soluble HLA ligand database stored on memory media associated with the computer system, the soluble HLA ligand database having soluble HLA ligand data stored therein; and
a data retrieval process that includes instructions for:
receiving a request from a request for general soluble HLA ligand data and returning the retrieved data to the request,
receiving a match request from the request for soluble HLA ligand data and returning data that matches the match request to the request, and p2 receiving a predictive request from the requestor for soluble HLA ligand data and returning data that matches the predictive request to the requester.

3. A soluble HLA ligand database assembled according to the methodology comprising the steps of:
providing a computer system capable of storing soluble HLA ligand data as a database on a memory media;
producing soluble HLA having ligands loaded thereon;
isolating the loaded ligands from the soluble HLA;
sequencing the loaded ligands to obtain soluble HLA ligand data; and
populating the database with the soluble HLA ligand data.

4. The soluble HLA ligand database of claim 3, further comprising the step of linearly manipulating the soluble HLA ligand data in the database.

5. The soluble HLA ligand database of claim 3, further comprising the step of manipulating the soluble HLA ligand data in the database with a predictive algorithm.
